(19) United States
(12) Patent Application Publication
(10) Pub. No.: US 2018/0207292 A1
(43) Pub. Date: Jul. 26, 2018

(54) METHODS FOR TREATING DISORDERS ASSOCIATED WITH ANGIOGENESIS AND NEOVASCULARIZATION

(71) Applicant: ICONIC THERAPEUTICS, INC., South San Francisco, CA (US)

(72) Inventors: Gabriela BURIAN, South San Francisco, CA (US); William GREENE, South San Francisco, CA (US); Kirk DORNBUSH, South San Francisco, CA (US)

(21) Appl. No.: 15/746,545

(22) PCT Filed: Jul. 22, 2016
(86) PCT No.: PCT/US16/43617

§ 371 Date: Jan. 22, 2018

Related U.S. Application Data
(60) Provisional application No. 62/195,709, filed on Jul. 22, 2015.

(51) Int. Cl. A61K 47/68; A61K 39/395; A61P 27/02; A61K 9/00; A61K 45/06; C07K 16/22

(52) U.S. Cl. CPC A61K 47/6815; A61K 39/3955; A61P 27/02; A61K 2039/545; A61K 45/06; A61K 9/0048; C07K 16/22; A61K 9/0019

(57) ABSTRACT

Provided herein are methods and immunoconjugate dimer compositions for the treatment of diseases associated with angiogenesis and neovascularization. In one aspect, the invention relates to a method for treating wet age-related macular degeneration (AMD) in an eye of a patient in need thereof. The method comprises administering to the patient in multiple dosing sessions, a composition comprising an effective amount of an immunoconjugate dimer, wherein the monomer subunits of the dimer each comprises a mutated human factor VIIa (fVIIa) protein conjugated to the human immunoglobulin G1 (IgG1) Fc domain.

### CROSS-REFERENCE TO RELATED APPLICATIONS

This application claims the benefit of U.S. Provisional Application Ser. No. 62/195,709, filed Jul. 22, 2015, which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ICTH_001_01WO.txt. The text file is 24 KB, was created on Jul. 18, 2016, and is being submitted electronically via EFS-Web.

### BACKGROUND OF THE INVENTION

Age-related macular degeneration (AMD) refers to the chronic, progressive degenerative pathology of the macula that results in loss of central vision. According to the Macula Vision Research Foundation and the National Eye Institute, as many as fifteen million people in the United States suffer from some form of AMD, with similar numbers in Europe and other continents. Neovascular AMD (also revered to as exudative or “wet” AMD) is the leading cause of severe vision loss and blindness in elderly patients over the age of fifty in the industrialized world. In the United States alone, more than 1.5 million people suffer from wet AMD. It is expected that AMD incidence and prevalence will further increase with the ageing population, thus leading to a significant increase in the number of patients with wet AMD in the United States and worldwide.

Tissue factor (IF) is a cytokine receptor present on vascular endothelial cells. It is an integral membrane glycoprotein with an intracellular terminal domain, a transmembrane domain, and an extracellular binding domain for Factor VII (FVII) and Factor VIIa (FVIIa). TF has been implicated in the process of angiogenesis and the inflammatory cascade of cytokine release, both processes in the pathogenesis of neovascular AMD and certain cancers.

Choroidal neovascularization (CNV) is the process in which new blood vessels grow in the choroid layer of the eye, and is associated with wet AMD. Therapies targeting vascular endothelial growth factor (VEGF) are currently the standard of clinical care for wet AMD. However, due to the multifaceted aspects of choroidal neovascularization and AMD pathogenesis, targeting VEGF alone is most likely insufficient to halt the progression of the disease towards the advanced CNV-associated degenerative processes.

There is an unmet medical need for new therapeutic strategies for choroidal neovascularization and age-related macular degeneration. The present invention addresses this and other needs.

### SUMMARY OF THE INVENTION

In one aspect, the present invention provides a method for treating wet age-related macular degeneration (AMD) in the an eye of a patient in need thereof, comprising, administering to the patient in a dosing regimen comprising multiple dosing sessions, a pharmaceutical composition comprising an effective amount of an immunoconjugate dimer comprising monomer subunits that each comprise a mutated human factor VIIa (fVIIa) protein conjugated to the human immunoglobulin G1 (IgG1) fragment crystallizable (Fc) region, or a portion thereof. In one embodiment, the mutated human fVIIa protein is conjugated to the human IgG1 via the hinge region of IgG1. In one embodiment, the immunoconjugate dimer is a homodimer. In another embodiment, the immunoconjugate dimer is a heterodimer. In one embodiment, the immunoconjugate dimer has the amino acid sequence of SEQ ID NO:2 or 3 In a further embodiment, the immunoconjugate dimer has the amino acid sequence of SEQ ID NO: 2. In one embodiment, the immunoconjugate dimer is encoded by SEQ ID NO:1, 4, or 5.

In one embodiment of the method for treating wet (AMD), each dosing session comprises intraocular injection e.g., intravitreal injection of the pharmaceutical composition. In another embodiment, each dosing session comprises topical administration of the pharmaceutical composition (e.g., via eye drops).

The multiple dosing sessions in one embodiment, comprise two or more, three or more, four or more or five or more dosing sessions. In a further embodiment, the time between each dosing session is from about 10 days to about 50 days, from about 10 days to about 40 days, from about 10 days to about 30 days or from about 10 days to about 20 days. In a further embodiment, the multiple dosing sessions comprise intravitreal injection of the pharmaceutical composition once every 14 days, once every 28 days, or once every 30 days.

In one embodiment, the method for treating wet age-related macular degeneration (AMD) comprises administering a pharmaceutical composition comprising an effective amount of the immunoconjugate dimer, wherein one or both of the monomer subunits comprises a mutated human factor VIIa having a substitution of alanine for lysine-341 (e.g., the protein of SEQ ID NO:2) or alanine for serine-344 (e.g, the protein of SEQ ID NO:3).

In one embodiment of the method for treating wet AMD in the eye of a patient in need thereof, the patient substantially maintains his or her vision subsequent to the multiple dosing sessions, as measured by losing fewer than 15 letters in a best-corrected visual acuity (BCVA) measurement, compared to the patient's BCVA measurement prior to the multiple dosing sessions. In a further embodiment, the loss of fewer than 15 letters in BCVA is sustained for at least about 10 days, at least about 20 days, at least about 30 days, at least about 40 days, at least about 50 days or at least about 100 days or at least one year after the treatment regimen has concluded. In another embodiment, the patient experiences an improvement in vision subsequent to the multiple dosing sessions, as measured by gaining 15 letters in a best-corrected visual acuity (BCVA) measurement, compared to the patient's BCVA prior to the multiple dosing sessions. In a further embodiment, the improvement in BCVA is sustained for at least about 10 days, at least about 20 days, at least about 30 days, at least about 40 days, at least about 50 days or at least about 100 days or at least one year after the treatment regimen has concluded.

In one embodiment of the method for treating wet AMD, subsequent to the multiple dosing sessions, or one or more of the dosing sessions, the CNV area is reduced in the eye of the patient, as compared to the CNV area prior to the multiple dosing sessions, or one or more of the dosing sessions (e.g., as measured by fluorescein angiography). In a further embodiment, the CNV area is reduced by at least about 10%, at least about 20% or at least about 30%, at least about 40% or at least about 50%, as compared to the CNV area prior to the multiple dosing sessions, or one or more dosing sessions.

In one embodiment of the method for treating wet AMD in the eye of the patient in need thereof, subsequent to the multiple dosing sessions, or a subset thereof, the retinal thickness of the eye of the patient is decreased as measured by optical coherence tomography (OCT), as compared to the retinal thickness of the eye prior to the multiple dosing sessions, or a subset thereof (e.g., the first dosing session, the first and second dosing session, etc.). The retinal thickness, in one embodiment, is a decreased by at least about 50 μm, at least about 100 μm, at least about 150 μm, at least about 175 μm, at least about 200 μm, at least about 225 μm, at least about 250 μm, at least about 275 μm or at least about 300 μm. The retinal thickness, in one embodiment, is a decreased by at least about 10%, at least about 20% or at least about 30%, as compared to retinal thickness of the eye prior to the multiple dosing sessions, or a subset thereof. The decreased retinal thickness in one embodiment is decreased central retinal subfield thickness (CST), decreased center point thickness (CPT), or decreased central foveal thickness (CFT).

In one embodiment, during treatment, or upon completion of treatment, neovascularization, e,g., choroidal neovascularization, of the eye of the patient is reversed. In another embodiment, neovascularization, e.g., choroidal neovascularization, of the eye of the patient is inhibited during the treatment regimen, or for at least about 10 days, at least about 20 days, at least about 30 days, at least about 40 days, at least about 50 days or at least about 100 days after the treatment regimen has concluded. In one embodiment, the patient in need of treatment has not been previously treated for wet AMD or choroidal neovascularization. However, in another embodiment, the patient has previously been treated for choroidal vascularization or wet AMD. In a further embodiment, the patient was non-responsive or not properly responsive to the previous treatment. In a further embodiment, the previous treatment for choroidal neovascularization or wet AMD comprises anti-vascular endothelial growth factor (VEGF) therapy, laser therapy or surgery.

In one embodiment, the method for treating wet age-related macular degeneration (AMD) comprises further administering a neovascularization inhibitor and/or angiogenesis inhibitor to the patient. In one embodiment, the neovascularization inhibitor and/or angiogenesis inhibitor is present in the same composition as the effective amount of the immunoconjugate dimer. However, in another embodiment, the neovascularization inhibitor and/or angiogenesis inhibitor is present in a different composition than the effective amount of the immunoconjugate dimer. In one embodiment, the neovascularization inhibitor and/or angiogenesis inhibitor is a vascular endothelial growth factor (VEGF) inhibitor, a VEGF receptor inhibitor, a platelet derived growth factor (PDGF) inhibitor or a PDGF receptor inhibitor.

Yet another embodiment of the method for treating wet AMD comprises administering to the patient in a dosing regimen comprising multiple intravitreal dosing sessions, a pharmaceutical composition comprising an effective amount of an immunoconjugate comprising a mutated human factor VIIa (fVIIa) protein conjugated to the human immunoglobulin G1 (IgG1) fragment crystallizable (Fc) region, or a portion thereof, and measuring the intraocular pressure (IOP) in the eye of the patient prior to and subsequent to each intravitreal injection, e.g., via tonometry. In a further embodiment, the method comprises measuring the IOP in the eye of the patient about 20 minutes, about 30 minutes, about 40 minutes, about 50 minutes or about 1 hour after each intravitreal injection.

In another aspect, the present invention provides a method for inhibiting, preventing or reversing ocular neovascularization in the an eye of a patient in need thereof, comprising, administering to the patient in a dosing regimen comprising multiple dosing sessions, a pharmaceutical composition comprising an effective amount of an immunoconjugate dimer comprising monomer subunits that each comprise a mutated human factor VIIa (fVIIa) protein conjugated to the human immunoglobulin G1 (IgG1) fragment crystallizable (Fe) region, or a portion thereof. In one embodiment of this aspect, the mutated human factor VIIa comprises a substitution of alanine for lysine-341 or of alanine for serine-344 (e.g., the immunoconjugate of SEQ ID NO: 2). In another embodiment, the ocular neovascularization is associated with (or secondary to) proliferative diabetic retinopathy, wet AMD, retinopathy of prematurity (ROP), or neovascular glaucoma. In a further embodiment, the ocular neovascularization is choroidal neovascularization.

In one embodiment of the method, after at least one dosing session of the pharmaceutical composition, choroidal neovascularization is inhibited for at least about 10 days, at least about 20 days, at least about 30 days, at least about 40 days, at least about 50 days or at least about 100 days after the at least one dosing session.

In the methods for inhibiting, preventing or reversing ocular neovascularization, multiple dosing sessions of the composition can be employed. For example, the pharmaceutical composition comprising the immunoconjugate dimer in one embodiment is administered two or more, three or more, four or more or five or more times to the patient in need thereof. In a further embodiment, the time between each dosing session is from about 10 days to about 50 days, from about 10 days to about 40 days, from about 10 days to about 30 days or from about 10 days to about 20 days. In a further embodiment, the multiple dosing sessions comprise intravitreal injection of the pharmaceutical composition once every 14 days, once every 28 days, or once every 30 days.

In one embodiment, the method for inhibiting, preventing or reversing ocular neovascularization comprises administering a pharmaceutical composition comprising an effective amount of the immunoconjugate dimer, wherein the mutated human factor VIIa comprises a substitution of alanine for lysine-341 (e.g., the protein of SEQ ID NO: 2) or of alanine for serine-344 (e.g., the protein of SEQ ID NO: 3). In one embodiment, the immunoconjugate is encoded by a polynucleotide sequence comprising SEQ ID NO:4 or SEQ ID NO:5.

In one embodiment of the method for inhibiting, preventing or reversing ocular neovascularization, the patient substantially maintains his or her vision subsequent to the multiple dosing sessions, as measured by losing fewer than 15 letters in a best-corrected visual acuity (BCVA) measurement, compared to the patient's BCVA measurement prior to the treatment (i.e., at least one dosing session). In a further embodiment, the loss of fewer than 15 letters in BCVA is sustained for at least about 10 days, at least about 20 days, at least about 30 days, at least about 40 days, at least about 50 days or at least about 100 days or at least one year after the treatment regimen (i.e., at least one dosing session) has concluded. In another embodiment, the patient experiences an improvement in vision subsequent to the multiple dosing sessions, as measured by gaining 15 letters in a best-corrected visual acuity (BCVA) measurement, compared to the patient's BCVA prior to initiation of treatment. In a further embodiment, the improvement in BCVA is sustained for at least about 10 days, at least about 20 days, at least about 30 days, at least about 40 days, at least about 50 days or at least about 100 days or at least one year after the treatment regimen(i.e., at least one dosing session) has concluded.

In another embodiment of the method for inhibiting, preventing or reversing ocular neovascularization, subsequent to administration of the pharmaceutical composition, as measured by fluorescein angiography, the CNV area is reduced in the eye of the patient, as compared to the CNV area prior to initiation of treatment. In one embodiment of the method for treating wet AMD in the eye of the patient in need thereof, subsequent to at least one dosing session, the retinal thickness of the eye of the patient is decreased as measured by optical coherence tomography (OCT), as compared to the retinal thickness prior to the initiation of treatment with the pharmaceutical composition. The decreased retinal thickness in one embodiment is decreased central retinal subfield thickness (CST), decreased center point thickness (CPT), or decreased central foveal thickness (CFT).

In some embodiments, administering the immunoconjugate comprises intravenous administration or intratumoral injection.

In one embodiment, each dosing session comprises the administration of between about 200 μg and about 400 μg of the immunoconjugate dimer. In a further embodiment, each dosing session comprises the administration of about 300 μg of the immunoconjugate dimer.

In one embodiment, a composition comprising an immunoconjugate dimer, wherein the monomer subunits of the dimer each comprises a mutated human factor VIIa (fVIIa) protein conjugated to the human immunoglobulin G1 (IgG1) Fc domain, for use in treating we age-related macular degeneration (AMD) in an eye of a patient in need thereof, wherein the composition is administered to the patient in multiple dosing sessions. In a further embodiment, further to the composition of use, treating the wet AMD comprises preventing, inhibiting, or reversing choroidal neovascularization in the eye of the patient in need of treatment.

In one embodiment, a composition comprising an immunoconjugate dimer, wherein the monomer subunits of the dimer each comprises a mutated human factor VIIa (fVIIa) protein conjugated to the human immunoglobulin G1 (IgG1) Fc domain, for use in preventing, inhibiting, or reversing ocular neovascularization in an eye of a patient in need thereof, wherein the composition is administered to the patient in multiple dosing sessions.

In one embodiment, a composition comprising an immunoconjugate dimer, wherein the monomer subunits of the dimer each comprises a mutated human factor VIIa (fVIIa) protein conjugated to the human immunoglobulin G1 (IgG1) Fc domain, for use in reversing tumor neovascularization in a patient in need thereof, wherein the composition is administered to the patient in multiple dosing sessions.

### BRIEF DESCRIPTION OF THE FIGURES

FIG. 1 is a non-limiting diagram of a general immunoconjugate embodiment of the present invention.

FIG. 2 is a graph of the rate of the intrinsic factor Xase complex (Fxase) hydrolysis (increase in absorbance at 405 nm−mOD/min) as a function of time.

FIG. 3 is a graph of thrombin generation by the known inhibitor of coagulation, active site inhibited FVIIa (FVIIai), as a function of time in normal pooled plasma.

FIG. 4 is a graph of thrombin generation by hI-con1, as a function of time in normal pooled plasma.

FIG. 5 is a graph of thrombin generation by human Factor VIIa and hI-con1 as a function of time in FVII-depleted plasma.

FIG. 6 is a graph of thrombin generation by hI-con1, as a function of time in rabbit plasma.

FIG. 7 is a graph of thrombin generation hI-con1 or FVIIai as a function of time in centrifuged rabbit plasma.

FIG. 8 is a graph showing the percent CNV in the pig as a function of intravitreal dose of hI-con1. Intravitreal injections (100 μL/eye) of solutions of hI-con1 (0,25, 0.5, 1.0 and 2.0 mg/mL) were injected into both eyes of mini-pigs on Day 10; control animals received 100 μL of formulation buffer. On Day 14 the animals were sacrificed and the % CNV was determined.

FIG. 9 is a graph showing the percent CNV in the pig as a function of intravitreal dose of a 100 kDa fragment of hI-con1. Intravitreal injections (100 μL/eye) of solutions of hI-con1 (0.25, 0.5, 1.0 and 2.0 mg/mL) were injected into both eyes of mini-pigs on Day 10; control animals received 100 μL of formulation buffer. On Day 14 the animals were sacrificed and the % CNV was determined.

### DETAILED DESCRIPTION OF THE INVENTION

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10%.

As used herein, “classic CNV” means a well-defined CNV area that results in vision that is between 20/250 and 20/400, but may be worse than 20/800.

As used herein, “occult CNV” means a poorly delineated CNV area that exhibits less leakage than classic CNV, and results in vision that is between 20/80 and 20/200.

### Angiogenesis

Pathologic angiogenesis, the induction of the growth of existing blood vessels from the vessels in surrounding tissue, is observed in a variety of diseases, typically triggered by the release of specific growth factors for vascular endothelial cells. Pathologic angiogenesis can result in neovascularization, i.e., the creation of new blood vessels, enabling solid tumor growth and metastasis, causing visual malfunction in ocular disorders, promoting leukocyte extravasation in inflammatory disorders, and/or influencing the outcome of cardiovascular diseases such as atherosclerosis.

In one aspect of the present invention, methods for treating a patient having a disease associated with neovascularization and/or angiogenesis, such as cancer, rheumatoid arthritis, the exudative (“wet”) form of macular degeneration, and/or atherosclerosis are provided. As described herein, administration may be local or systemic, depending upon the type of pathological condition involved in the therapy. As used herein, the term “patient” includes both humans and other species, including other mammal species. The invention thus has both medical and veterinary applications. In veterinary compositions and treatments, immunoconjugate are constructed using targeting and effector domains derived from the corresponding species.

The present invention is based in part on the observation that normal adult mammalian vasculature is generally in a quiescent state (except for certain processes such as the female reproductive cycle and wound healing), in contrast to the neovasculature that forms in certain disease states such as choroidal neovascularization or a growing tumor which is in an active state of angiogenesis. Therefore, a molecular difference between quiescent and proliferating vascular endothelial cells could serve as a target for the pathologic vasculature. One molecular difference between quiescent and proliferating vascular endothelial cells is that the latter express tissue factor, upon binding of VEGF to its respective cell surface receptor. Tissue factor is a transmembrane receptor that binds plasma factor VII/VIIa to initiate blood coagulation. Because only the vascular endothelial cells that have bound VEGF express tissue factor, a putative target for activated vasculature (e.g., tumor vasculature) is tissue factor expressed on endothelial cells.

In the aspects provided herein, methods for treating a patient for a disease associated with angiogenesis and/or neovascularization are provided. In one embodiment, the disease associated with neovascularization and/or angiogenesis is wet AMD. In another embodiment, the disease associated with neovascularization and/or angiogenesis is a cancer.

As used herein, “immunoconjugate” or “immunoconjugate” refer to a conjugate protein such as ICON-1. In some embodiments, an immunoconjugate has as an effector domain an immunoglobulin Fc domain, and said effector domain is conjugated to a targeting domain comprising a mutant form of human factor VII. In some embodiments, an immunoconjugate comprises an Fc domain of a human IgG1 immunoglobulin conjugated to a targeting domain comprising a mutant form of factor VII comprising one or two mutations selected from S344A and/or K341A, wherein the immunoconjugate protein binds to tissue factor. In some embodiments, immunoconjugates of the present disclosure include immunoconjugates described in U.S. Pat. Nos. 7,858,092; 8,388,974, 8,071,104; 7,887,809; and 6,924,359.

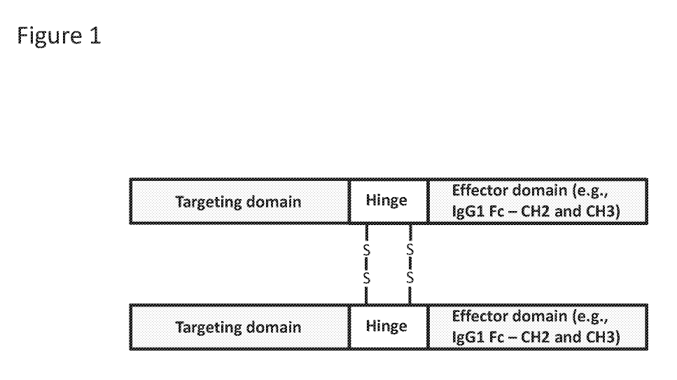

In one aspect provided herein, a composition comprising a fusion protein comprising a mutated FVII protein (targeting domain) conjugated to a human IgG1 Fc region (effector domain) are provided. FIG. 1 provides the generalized structure of one embodiment of an immunoconjugate that can be administered by the methods provided herein. The mutated Factor VIIa domain (also referred to as the TF targeting domain), in the aspects provided herein, binds tissue factor with high affinity and specificity, but does not initiate coagulation, or minimizes coagulation normally associated with tissue factor binding. The IgG1 Fc domain (also referred to as the effector domain) triggers a cytolytic response against cells which bind the immunoconjugate, by the natural killer (NK) cell and complement pathways. In one embodiment, the IgG1 Fe effector domain comprises both the CH2 and CH3 regions of the IgG1 Fc region.

TABLE 1
Description of Sequences

| Sequence Identifier | Description |
|---|---|
| SEQ ID NO: 1 | Homo sapiens factor VII active site mutant |
| | immunoconjugate mRNA, complete CDS; |
| | NCBI Accession AF272774 |
| SEQ ID NO: 2 | Homo sapiens factor VII active site mutant |
| | immunoconjugate amino acid sequence |
| SEQ ID NO: 3 | Homo sapiens factor VII active site mutant |
| | immunoconjugate amino acid sequence, |
| | S344A and A341K (relative to SEQ ID NO: 2) |
| SEQ ID NO: 4 | Homo sapiens factor VII active site mutant |
| | immunoconjugate coding sequence |
| SEQ ID NO: 5 | Homo sapiens factor VII active site mutant |
| | immunoconjugate coding sequence |

The reaction between FVIIa and TF is species-specific (Janson et al., 1984; Schreiber et al., 2005; Peterson et al., 2005): murine FVII appears to be active in many heterologous species including rabbit, pig and human, whereas human FVIIa is only appreciably active in human, dog, rabbit and pig. Conversely, the human IgG Fc domain is active in both humans and mice. Accordingly, depending on the patient, the immunoconjugate is constructed using targeting and effector domains derived from the corresponding species, or from a species that is known to be active in the patient. For example, in the human treatment methods provided herein, the mutated tissue factor targeting domain is derived from human Factor VIIa conjugated to an effector domain comprising the Fc region of a human IgG1 immunoglobulin. For example, in one embodiment, the immunoconjugate is a protein of SEQ ID NO: 2. In a further embodiment, the immunoconjugate is a protein of SEQ ID NO: 3. In one embodiment, the immunoconjugate is encoded by the mRNA sequence of SEQ ID NO: 1, 4, or 5.

In one embodiment, the immunoconjugate described herein comprises two protein chains, each comprising a targeting domain joined to an effector domain via a linker or hinge region. In a further embodiment, the linker or hinge region is naturally occurring, and in one embodiment, is of human origin. The hinge region of an IgG1 immunoglobulin, for example the hinge region of the human IgG1 immunoglobulin, in one embodiment, is used to link the targeting domain to the effector domain. In one embodiment, the hinge region of IgG1 includes cysteine amino acids which form one or more disulfide bonds between the two monomer chains (e.g., as depicted in FIG. 1).

In one embodiment, the immunoconjugate is a homodimer. However, in another embodiment, the immunoconjugate is a heterodimer, for example, an immunoconjugate comprising two monomers each having a targeting domain of a different amino acid sequence, but the same effector domains. The amino acid sequences of the two targeting domains, in one embodiment, differ by one amino acid, two or more amino acids, three or more amino acids or five or more amino acids. In one embodiment, each monomer subunit comprises an IgG1 hinge region that links the targeting region and effector region of the immunoconjugate, and the monomer subunits of the immunoconjugate heterodimer or the immunoconjugate homodimer are linked together via a disulfide bond between IgG1 hinge regions.

In one embodiment, the molecular weight of the immunoconjugate provided herein is from about 150 kDa to about 200 kDa. In another embodiment, the molecular weight of the immunoconjugate is about 157 kDa or 157 kDa. For example, the immunoconjugate in one embodiment is the immunoconjugate having the amino acid sequence set forth in SEQ ID NO: 2, also referred to herein as “hI-con1” or “ICON-1” In another embodiment, the immunoconjugate has the amino acid sequence set forth in SEQ ID NO: 3.

As provided throughout, in embodiments described herein, an immunoconjugate comprising a tissue factor targeting domain comprising a mutated Factor VIIa domain is provided. The targeting domain comprises a mutated Factor VIIa that has been mutated to inhibit initiation of the coagulation pathway without reducing binding affinity to tissue factor. In one embodiment, the mutation in Factor VIIa is a single point mutation at residue 341. In a further embodiment, the mutation is from Lys341 to Ala341. However, other mutations that inhibit the coagulation pathway are encompassed by the immunoconjugates provided herein. The effector domain of the immunoconjugates provided herein, in one embodiment, mediates both complement and natural killer (NK) cell cytotoxicity pathways.

### Immunoconjugate Production

In some embodiments, methods of producing the immunoconjugate include expression in mammalian cells such as BHK cells. In further embodiments, cell lines may include HEK 293, CHO, and SP2/0. Immunoconjugates may be generated by mammalian expression of the expression constructs. In some embodiments, the immunoconjugates are produced as fusion proteins (FVII-Fc) or produced as chemical conjugates.

In some embodiments, the immunoconjugate is post-translationally modified. Post-translational modification includes: myristoylation, glypiation, palmitoylation, prenylation, lipoylation, acylation, alkylation, butrylation, gamma-carboxylation, glycosylation (N-glycosylation, O-glycosylation, fucosylation, and mannosylation), propionylation, succinylation, and sulfation.

### Administering the Immunoconjugate

Administration methods encompassed by the methods provided herein include intravitreal injection, suprachoroidal injection, topical administration (e.g., eye drops), intravenous and intratumoral administration. In another embodiment, administration is via intravenous, intramuscular, intratumoral, subcutaneous, intrasynovial, intraocular, intraplaque, or intradermal injection of the immunoconjugate or of a replication-deficient adenoviral vector, or other viral vectors carrying a cDNA encoding a secreted form of the immunoconjugate. In one embodiment, the patient in need of treatment is administered one or more immunoconjugate dimers via intravitreal, intravenous or intratumoral injection, or injection at other sites, of one or more immunoconjugate proteins. Alternatively, in one embodiment, a patient in need of treatment is administered one or more immunoconjugate dimers via intravenous or intratumoral injection, or injection at other sites, of one or more expression vectors carrying a cDNA encoding a secreted form of one or more of the immunoconjugate dimers provided herein. In some embodiments, the patient is treated by intravenous or intratumoral injection of an effective amount of one or more replication-deficient adenoviral vectors, or one or more adeno-associated vectors carrying cDNA encoding a secreted form of one or more types of immunoconjugate proteins.

As used herein, “effective amount” or “therapeutically effective amount” means a level or amount of a therapeutic agent needed to treat a condition or disease of the present disclosure, or the level or amount of a therapeutic agent that produces a therapeutic response or desired effect in the subject to which the therapeutic agent was administered; wherein a therapeutic agent is an immunoconjugate of the present disclosure. Thus, a therapeutically effective amount of a therapeutic agent, such as an immunoconjugate of the present disclosure, is an amount that is effective in reducing one or more symptoms of angiogenesis and/or neovascularization, as well as various forms of AMD.

As used herein, “pharmaceutical composition” means a composition comprising a therapeutic agent.

As used herein, “treatment”, “treating”, and the like, mean the following actions: (i) preventing a particular disease or disorder from occurring in a subject who may be predisposed to the disease or disorder but has not yet been diagnosed as having it; (ii) curing, treating, or inhibiting the disease, i.e., arresting its development; or (iii) ameliorating the disease by reducing or eliminating symptoms, conditions, and/or by causing regression of the disease.

In one embodiment, a method of intravitreal injection is employed. In a further embodiment, aseptic technique is employed when preparing the immunoconjugate dimer for injection, for example, via the use of sterile gloves, a sterile drape and a sterile eyelid speculum (or equivalent). In one embodiment, the patient is subjected to anesthesia and a broad-spectrum microbicide prior to the injection.

In one embodiment, intravitreal injection of one or more of the immunoconjugate dimers provided herein, for example the immunoconjugate dimer of SEQ ID NO: 2 is prepared by withdrawing the vial contents of the immunoconjugate dimer composition solution through a 5-micron, 19-gauge filter needle attached to a 1-cc tuberculin syringe. The filter needle in a further embodiment, is then discarded and replaced with a sterile 30-gauge×½-inch needle for the intravitreal injection. The contents of the vial are expelled until the plunger tip is aligned with the line on the syringe that marks the appropriate dose for delivery.

In one method of ocular injection, e,g., intravitreal or suprachoroidal injection, prior to and/or after the injection, the patient is monitored for elevation in intraocular pressure (IOP). For example, in one embodiment, prior to and/or after the ocular injection, the patient is monitored for elevation in IOP using tonometry. In another embodiment, the patient is monitored for increases in IOP via a check for perfusion of the optic nerve head immediately after the injection. In one embodiment, prior to ocular injection of one of the immunoconjugate dimers provided herein, for example about 20 minutes, about 30 minutes, about 40 minutes, about 50 minutes or about 1 hour prior to the ocular injection, the patient is monitored for elevation in IOP. In another embodiment, after ocular injection of one of the immunoconjugate dimers provided herein, for example, about 10 minutes, about 20 minutes, about 30 minutes, about 40 minutes, about 50 minutes or about 1 hour after the intraocular injection, the patient is monitored for elevation in IOP. In one embodiment, the patient's IOP is substantially the same prior to intraocular injection of an immunoconjugate dimer, as compared to after intraocular injection of the immunoconjugate dimer. In one embodiment, the patient's IOP varies by no more than 10%, no more than 20% or no more than 30% after intraocular injection, as compared to prior to intraocular injection (e.g., intravitreal injection).

The treatment methods provided herein in one embodiment, comprise a single administration of one of the immunoconjugate dimers provided herein (e.g., an immunoconjugate of SEQ ID NO: 2 or 3). However, in another embodiment, the treatment methods provided herein comprise multiple dosing sessions. In a further embodiment, the multiple dosing sessions are multiple intraocular injections of one of the immunoconjugate dimers described herein. The multiple dosing sessions, in one embodiment comprise two or more, three or more, four or more or five or more dosing sessions. In a further embodiment, each dosing session comprises intraocular injection of one of the immunoconjugates described herein, or intratumoral injection of one of the immunoconjugates described herein (i.e., either as the expressed protein or via a vector encoding the soluble immunoconjugate).

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed.

In one embodiment, where multiple dosing sessions are employed, the dosing sessions are spaced apart by from about 10 days to about 60 days, or from about 10 days to about 50 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In even another embodiment, the multiple dosing sessions are bi-weekly (e.g., about every 14 days), monthly (e.g., about every 30 days), or hi-monthly (e.g., about every 60 days). In yet another embodiment, the dosing sessions are spaced apart by about 28 days.

In one embodiment, the multiple dosing sessions comprise 2, 3, 4, 5, 6, 7, 8, 9, 10, 11, 12, 13, 14, 15, 16, 17, 18, 19, 20, 21, 22 23, 24, 25, 26, 27, 28, 29, 30, 31, 32, 33, 34, 35, 36, 37, 38, 39, 40, 41, 42, 43, 44, 45, 46, 47, 48, 49, or 50 dosing sessions, wherein the dosing sessions are spaced apart by 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, or 60 days.

The immunoconjugates provided herein are amenable for use in any disease or disorder in which angiogenesis and/or neovascularization is implicated. For example, in one aspect, an immunoconjugate dimer provided herein is administered to the eye of a patient in need of treatment of wet age-related macular degeneration (AMD). In one embodiment, the treatment comprises multiple dosing sessions of the immunoconjugate dimer. As provided throughout, the immunoconjugate dimer comprises monomer subunits that each include a mutated human factor VIIa (fVIIa) protein conjugated to the human immunoglobulin G1 (IgG1) Fc domain. In a further embodiment, the immunoconjugate dimer has the amino acid sequence of SEQ ID NO: 2 or 3. In a further embodiment, the immunoconjugate dimer has the amino acid sequence of SEQ ID NO: 3.

In one embodiment, the method of treating wet AMD comprises preventing, inhibiting or reversing choroidal neovascularization in the eye of the patient in need of treatment. In a further embodiment, choroidal neovascularization is reversed by at least about 10%, at least about 20%, at least about 30% or at least about 40% after treatment, as compared to the choroidal neovascularization that was present in the afflicted eye of the patient prior to treatment.

Other ocular disorders associated with ocular neovascularization are treatable with the immunoconjugates and methods provided herein. The ocular neovascularization, in one embodiment, is choroidal neovascularization. In another embodiment the ocular neovascularization is retinal neovascularization. In yet another embodiment, the ocular neovascularization is corneal neovascularization. Accordingly, an ocular disorder associated with choroidal, retinal or corneal neovascularization, in one embodiment, is treatable by one or more of the methods provided herein. In a further embodiment, the method comprises administering to the eye of a patient in need thereof, one of the immunoconjugate dimers described herein. In a further embodiment, the treatment comprises multiple dosing sessions of the immunoconjugate dimer. In a further embodiment, the immunoconjugate dimer has the amino acid sequence of SEQ NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3.

For example, in one embodiment, a patient in need of treatment of proliferative diabetic retinopathy, wet age-related macular degeneration (AMD), retinopathy of prematurity (ROP), or neovascular glaucoma is treated with one of the immunoconjugates provided herein, for example, via intravitreal injection, suprachoroidal injection or topical administration (e.g., via eye drops) of the immunoconjugate into the affected eye. Treatment in one embodiment occurs over multiple dosing sessions. With respect to the aforementioned disorders, ocular neovascularization is said to be “associated with” or “secondary to” the respective disorder.

In one embodiment, a patient in need of treatment of macular edema following retinal vein occlusion (RVO) is treated by one of the immunoconjugate dimers provided herein. In one embodiment, the method comprises administering to the patient a composition comprising an effective amount of an immunoconjugate dimer, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dialer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions, for example, via intravitreal administration at each dosing session.

In another embodiment, a patient in need of treatment of diabetic macular edema (DME) is treated by one of the immunoconjugate dimers provided herein. In one embodiment, the method comprises administering to the patient a composition comprising an effective amount of an immunoconjugate dimer, wherein the monomer subunits of the dimer each comprise a mutated factor VIIa (fVIIa) protein conjugated to the human immunoglobulin (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions. In even a further embodiment, the immunoconjugate dimer is administered intravitreally at each dosing session.

In yet another embodiment, diabetic retinopathy is treated via one of the immunoconjugates provided herein, in a patient in need thereof, for example, a patient with DME. In one embodiment, the method comprises administering to the patient, for example a DME patient, a composition comprising an effective amount of an immunoconjugate dimer, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions. In even a further embodiment, the immunoconjugate dimer is administered to the patient over multiple dosing sessions, for example, via intravitreal administration at each dosing session.

In one embodiment of the invention, one or more of the immunoconjugates provided herein is used in a method to treat a disease or disorder associated with tumor neovascularization in a patient in need thereof, for example, a cancer patient. In one embodiment, the method comprises administering to the patient, for example via intratumoral or intravenous injection, a composition comprising an effective amount of an immunoconjugate dimer, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions.

In cancer treatments, the immunoconjugate dimer is used for treating a variety of cancers, particularly primary or metastatic solid tumors, including melanoma, renal, prostate, breast, ovarian, brain, neuroblastoma, head and neck, pancreatic, bladder, endometrial and lung cancer. In one embodiment, the cancer is a gynecological cancer. In a further embodiment, the gynecological cancer is serous, clear cell, endometriod or undifferentiated ovarian cancer. The immunoconjugate dimer in one embodiment is employed to target the tumor vasculature, particularly vascular endothelial cells, and/or tumor cells. Without wishing to be bound by theory, targeting the tumor vasculature offers several advantages for cancer immunotherapy with one or more of the immunoconjugate dimers described herein, as follows. (i) some of the vascular targets including tissue factor should be the same for all tumors; (ii) immunoconjugates targeted to the vasculature do not have to infiltrate a tumor mass in order to reach their targets; (iii) targeting the tumor vasculature should generate an amplified therapeutic response, because each blood vessel nourishes numerous tumor cells whose viability is dependent on the functional integrity of the vessel; and (iv) the vasculature is unlikely to develop resistance to an immunoconjugate, because that would require modification of the entire endothelium layer lining a vessel. Unlike previously described antiangiogenic methods that inhibit new vascular growth, immunoconjugate dimers provided herein elicit a cytolytic response to the neovasculature.

In another embodiment, one or more of the immunoconjugates described herein is used in a method for treating atherosclerosis or rheumatoid arthritis. In one embodiment, the method comprises administering to the patient in need of treatment a composition comprising an effective amount of an immunoconjugate dimer, wherein the monomer subunits of the dimer each comprise a mutated factor VIIa (fVIIa) protein conjugated to the human immunoglobulin G1 (IgG1) Fc domain. In a further embodiment, the mutated fVIIa protein is a human mutated fVIIa protein and is linked to the IgG1 Fc domain via the hinge region of IgG1. In a further embodiment, the immunoconjugate dimer has the amino acid sequence of SEQ ID NO: 2 or 3. In yet a further embodiment, the immunoconjugate dimer has the amino acid sequence of SEQ ID NO: 2. In a further embodiment, the immunoconjugate dimer has the amino acid sequence of SEQ ID NO: 3. In one embodiment, the immunoconjugate dimer is administered to the patient over multiple dosing sessions.

In one embodiment of a method for treating an ocular disorder with an immunoconjugate dimer, for example, a method for treating wet AMD, diabetic retinopathy, diabetic macular edema, or choroidal neovascularization secondary to an ocular disorder such as wet AMD, the patient subjected to the treatment method substantially maintains his or her vision subsequent to the treatment (e.g., the single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

In some embodiments, a patient having been administered an immunoconjugate of the present invention loses fewer than 10, 9, 8, 7. 6, or 5 letters in a BCVA measurement, compared to a patient's BCVA measurement prior to undergoing treatment. In some embodiments, the patient loses fewer than about 10, about 9, about 8, about 7, about 6, or about 5 letters in a BCVA measurement, compared to a patient's BCVA measurement prior to undergoing treatment.

In some embodiments, a patient having been administered an immunoconjugate of the present invention loses fewer than between 15 and 5, 15 and 6, 15 and 7, 15 and 8, 15 and 9, 15 and 10, 10 and 5, 10 and 6, 10 and 7, 10 and 8, 10 and 9, 9 and 5, 9 and 6, 9 and 7, 9 and 8, 8 and 5, 8 and 6, 8 and 7, 7 and 5, 7 and 6, or 6 and 5 letters in a BCVA measurement.

In some embodiments, a patient having been administered an immunoconjugate of the present invention loses fewer than between about 15 and about 5, about 15 and about 6, about 15 and about 7, about 15 and about 8, about 15 and about 9, about 15 and about 10, about 10 and about 5, about 10 and about 6, about 10 and about 7, about 10 and about 8, about 10 and about 9, about 9 and about 5, about 9 and about 6, about 9 and about 7, about 9 and about 8, about 8 and about 5, about 8 and about 6, about 8 and about 7, about 7 and about 5, about 7 and about 6, or about 6 and about 5 letters in a BCVA measurement.

In another embodiment of a method for treating an ocular disorder with an immunoconjugate dimer, for example, a method for treating wet AMD, diabetic retinopathy, diabetic macular edema, or choroidal neovascularization secondary to an ocular disorder such as wet AMD, the patient subjected to the treatment method substantially maintains his or her vision subsequent to the treatment (e.g., the single dosing session or multiple dosing sessions), as measured by BCVA measurement.

In some embodiments, a patient having been administered an immunoconjugate of the present invention regains his or her vision subsequent to the treatment, as measured by gaining 5, 6, 7, 8, 9, 10, 15, 20, or 25 or more letters in a best-corrected visual acuity (BCVA) measurement, compared to the patient's BCVA prior to the treatment. In some embodiments, a patient having been administered an immunoconjugate of the present invention regains his or her vision subsequent to the treatment, as measured by gaining about 5, about 6, about 7, about 8, about 9, about 10, about 15, about 20, or about 25 or more letters in a best-corrected visual acuity (BCVA) measurement, compared to the patient's BCVA prior to the treatment.

In some embodiments, a patient having been administered an immunoconjugate of the present invention regains his or her vision subsequent to the treatment, as measured by gaining greater than between 5 and 25, 5 and 20, 5 and 15, 5 and 10, 5 and 9, 5 and 8, 5 and 7, 5 and 6, 6 and 25, 6 and 20, 6 and 15, 6 and 10, 6 and 9, 6 and 8, 6 and 7, 7 and 25, 7 and 20, 7 and 15, 7 and 10, 7 and 9, 7 and 8, 8 and 25, 8 and 20, 8 and 15, 8 and 10, 8 and 9, 9 and 25, 9 and 20, 9 and 15, 9 and 10, 10 and 25, 10 and 20, 10 and 15, 1.5 and 25, 15 and 20, or 20 and 25 or more letters in a BCVA measurement, compared to the patient's BCVA prior to the treatment.

In some embodiments, a patient having been administered an immunoconjugate of the present invention regains his or her vision subsequent to the treatment, as measured by gaining greater than between about 5 and about 25, about 5 and about 20, about 5 and about 15, about 5 and about 10, about 5 and about 9, about 5 and about 8, about 5 and about 7, about 5 and about 6, about 6 and about 25, about 6 and about 20, about 6 and about 15, about 6 and about 10, about 6 and about 9, about 6 and about 8, about 6 and about 7, about 7 and about 25, about 7 and about 20, about 7 and about 15, about 7 and about 10, about 7 and about 9, about 7 and about 8, about 8 and about 25, about 8 and about 20, about 8 and about 15, about 8 and about 10, about 8 and about 9, about 9 and about 25, about 9 and about 20, about 9 and about 15, about 9 and about 10, about 10 and about 25, about 10 and about 20, about 10 and about 15, about 15 and about 25, about 15 and about 20, or about 20 and about 25 or more letters in a BCVA measurement, compared to the patient's BCVA prior to the treatment.

In one embodiment of a method for treating an ocular disorder with an immunoconjugate dimer, for example, a method for treating wet AMD, diabetic retinopathy, diabetic macular edema, or choroidal neovascularization secondary to an ocular disorder such as wet AMD, the ocular neovascularization area, e.g., the choroidal neovascularization area is reduced in the eye of the patient, as compared to the ocular neovascularization area (e.g., CNV area) prior to treatment. As provided herein, treatment can include one dosing session or multiple dosing sessions, and reduction in ocular neovascularization area (e.g., CNV area), in one embodiment, is assessed after individual dosing sessions, or multiple dosing sessions. In a further embodiment, the ocular neovascularization area (e.g., CNV area) is reduced by at least about 5%, or at least about 10%, or at least about 15%, or at least about 20%, or at least about 25%, or at least about 30%, or at least about 35%, or at least about 40%, or at least about 45%, or at least about 50%, as measured by fluorescein angiography.

In one embodiment of a method for treating an ocular disorder with an immunoconjugate dimer, for example, a method for treating wet AMD, diabetic retinopathy, diabetic macular edema, or choroidal neovascularization secondary to an ocular disorder such as wet AMD, the retinal thickness of the treated eye is reduced in the eye of the patient, as compared to the retinal thickness prior to treatment, as measured by optical coherence tomography (OCT). As provided herein, treatment can include one dosing session or multiple dosing sessions, and reduction in retinal thickness, in one embodiment, is assessed after individual dosing sessions, or multiple dosing sessions. In a further embodiment, the retinal thickness is reduced by at least about 5%, or at least about 10%, or at least about 15%, or at least about 20%, or at least about 25%, or at least about 30%, or at least about 35%, or at least about 40%, or at least about 45%, or at least about 50%, as measured by OCT. In a further embodiment, the decreased retinal thickness is decreased central retinal subfield thickness (CST), decreased center point thickness (CPT), or decreased central foveal thickness (CFT).

In one embodiment, the immunoconjugate dimer is administered as a solution or a suspension. The immunoconjugate composition, in one embodiment, comprises arginine or protein A. In a further embodiment, the immunoconjugate composition comprises arginine. In even a further embodiment, the arginine is present in the composition at from about 20 mM to about 40 mM, e.g., at 25 mM. Other components of the composition, in one embodiment, include HEPES, sodium chloride, polysorbate-80, calcium chloride, or a combination thereof.

In one embodiment, the immunoconjugate dimer is administered in a dose of between 10 μg and 500 μg, 10 μg and 400 μg, 10μg and 300 μg, 10 μg and 200 μg, 10 μg and 100 μg, 10 μg and 50 μg, 50 μg and 500 μg, 50 μg and 400 μg, 50 μg and 300 μg, 50 μg and 200 μg, 50 μg and 100μg, 100 μg and 500 μg, 100 μg and 400 μg, 100 μg and 300 μg, 100 μg and 200 μg, 200 μg and 500 μg, 200 μg and 400 μg, 200 μg and 300 μg, 300 μg and 500 μg, 300 μg and 400 μg, or 400 μg and 500 μg.

In one embodiment, the immunoconjugate dimer is administered in a dose of between about 10 μg and about 500 μg, about 10 μg and about 400 μg, about 10μg and about 300 μg, about 10 μg and about 200 μg, about 10 μg and about 100 μg, about 10 μg and about 50 μg, about 50 μg and about 500 μg, about 50 μg and about 400 μg, about 50 μg and about 300 μg, about 50 μg and about 200 μg, about 50 μg and about 100μg, about 100 μg and about 500 μg, about 100 μg and about 400 μg, about 100 μg and about 300 μg about 100 μg and about 200 μg, about 200 μg and about 500 μg, about 200 μg and about 400 μg, about 200 μg and about 300 μg, about 300 μg and about 500 μg, about 300 μg and about 400 μg, or about 400 μg and about 500 μg.

In one embodiment, the immunoconjugate dimer is administered in a dose consisting of about 10 μg, about 20 μg, about 30 μg, about 40 μg, about 50 μg, about 60 μg, about 70 μg about 80 μg, about 90 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, or about 700 μg,

In one embodiment, the immunoconjugate dimer is administered in a solute volume of between 10 μL and 200 μL, 10 μL, and 180 μL, 10 μL, and 160 μL, 10 μL and 140 μL, 10 μL and 120 μL, 10 μL and 100 μL, 10 μL and 80 μL, 10 μL and 60 μL, 10 μL and 40 μL, 10 μL and 20 μL, 10 μL and 15 μL, 20 μL and 200 μL, 20 μL and 180 μL, 20 μL and 160 μL, 20 μL and 140 μL, 20 μL and 120 μL, 20 μL and 100 μL, 20 μL and 80 μL, 20 μL and 60 μL, 20 μL and 40 μL, 40 μL and 200 μL, 40 μL and 180 μL, 40 μL and 160 μL, 40 μL and 140 μL, 40 μL and 120 μL, 40 μL and 100 μL, 40 μL and 80 μL, 40 μL and 60 μL, 60 μL and 200 μL, 60 μL and 180 μL, 60 μL and 160 μL, 60 μL and 140 μL, 60 μL and 120 μL, 60 μL and 100 μL, 60 μL and 80 μL, 80 μL and 200 μL, 80 μL and 180 μL, 80 μL and 160 μL, 80 μL and 140 μL, 80 μL and 120 μL, 80 μL and 100 μL, 100 μL and 200 μL, 100 μL and 180 μL, 100 μL and 160 μL, 100 μL and 140 μL, 100 μL and 120 μL, 120 μL and 200 μL, 120 μL and 180 μL, 120 μL and 160 μL, 120 μL and 140 μL, 140 μL and 200 μL, 140 μL, and 180 μL, 140 μL and 160 μL, 160 μL and 200 μL, 160 μL, and 180 μL, or 180 μL and 200 μL.

In one embodiment, the immunoconjugate dimer is administered in a solute volume consisting of about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL about 80 μL, about 85 μL, about 90 μL, about 95 μL, or about 100 μL.

One exemplary composition of the present invention is provided in Table 2 below.

TABLE 2
Exemplary immunoconjugate dimer composition of the invention

| Component | Concentration |
|---|---|
| Immunoconjugate dimer | 3 mg/mL in 15 mM HEPES |
| NaCl | 150 mM |
| Arginine | 25 mM, pH 7.4 |
| Polysorbate-80 | 0.01% |
| CaCl₂ | 5 mM |

### Co-Therapeutic Administration

The immunoconjugate dimer described herein in one embodiment is administered in a co-therapeutic regimen to treat a patient for one of the aforementioned diseases or disorders, for example, to treat wet AMD or another ocular disease associated with angiogenesis or neovascularization. The second active agent in one embodiment, is administered in the same composition as the immunoconjugate dimer. However, in another embodiment, the immunoconjugate dimer is administered in a separate composition. In one embodiment, the second active agent is a neovascularization inhibitor or an angiogenesis inhibitor.

The angiogenesis or neovascularization inhibitor, in one embodiment, is a vascular endothelial growth factor (VEGF) inhibitor, a VEGF receptor inhibitor, a platelet derived growth factor (PDGF) inhibitor or a PDGF receptor inhibitor.

In another embodiment, the neovascularization inhibitor is an integrin antagonist, a selectin antagonist, an adhesion molecule antagonist (e.g., antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1)), a basic fibroblast growth factor antagonist, a vascular endothelial growth factor (VEGF) modulator, or a platelet derived growth factor (PDGF) modulator (e.g., a PDGF antagonist). In one embodiment of determining whether a subject is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-1β), monocyte chemotactic protein-1 (MCP-1) and a vascular endothelial growth factor (VEGF)), as described in U.S. Pat. No. 6,524,581, incorporated by reference in its entirety herein.

In another embodiment, the neovascularization inhibitor is one or more of the following angiogenesis inhibitors: interferon gamma 1β, interferon gamma 1β (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RX1109, secretin, STX100, TGF-β Inhibitor, transforming growth factor, β-receptor 2 oligonucleotide, VA999260, XV615, or a combination thereof.

In another embodiment, the neovascularization inhibitor is an endogenous angiogenesis inhibitors. In a further embodiment, the endogenous angiogenesis inhibitor is endostatin, a 20 kDa C-terminal fragment derived from type XVIII collagen, angiostatin (a 38 kDa fragment of plasmin), or a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g,, soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN) (e.g., IFN-α, IFN-β, IFN-γ), a chemokine, e.g., a chemokine having the C-X-C motif (e.g., CXCL10, also known as interferon gamma-induced protein 10 or small inducible cytokine B10), an interleukin cytokine (e.g., IL-4, IL-12, IL,-18), prothrombin, antithrombin III fragment, prolactin, the protein encoded by the TNFSFI5 gene, osteopontin, maspin, canstatin, proliferin-related protein.

In one embodiment, one or more of the following neovascularization inhibitors is administered with the immunoconjugate described herein: angiopoietin-1, angiopoietin-2, angiostatin, endostatin, vasostatin, thrombospondin, calreticulin, platelet factor-4, TIMP, CDAI interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1, TSP-2, interferon gamma β, ACUHTR028, αVβ5, amino⁻benzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide,VA999260, XV615 or a combination thereof.

Yet another co-therapy embodiment includes administration of one of the immunoconjugates described herein with one or more of the following: pazopanib (Votrient), sunitinib (Sutent), sorafenib (Nexavar), axitinib (Inlyta), ponatinib (Iclusig), vandetanib (Caprelsa), cabozantinib (Cometrig), bevacizumab (Avastin), ramucirumab (Cyramza), regorafenib (Stivarga), ziv-aflibercept (Zaltrap), or a combination thereof. In yet another embodiment, the angiogenesis inhibitor is a VEGF inhibitor. In a further embodiment, the VEGF inhibitor is axitinib, cabozantinib, aflibercept, brivanib, tivozanib, ramucirumab or motesanib.

In one embodiment, the angiogenesis inhibitor is ranibizumab or bevacizumab. In a further embodiment, the angiogenesis in inhibitor is ranibizumab. In even a further embodiment, ranibizumab is administered at a dosage of 0.5 mg or 0.3 mg per dosing session, and is administered as indicated in the prescribing information for LUCENTIS.

In one embodiment, the co-therapy comprises administration of an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-α or PDGFR-β. In one embodiment, the PDGF antagonist is the anti-PDGF-β aptamer E10030, sunitinib, axitinib, sorefenib, imatinib, imatinib mesylate, nintedanib, pazopanib HCl, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

### Treatment Outcomes

In one embodiment, a BCVA letter score is determined in a patient or a population of patients wherein patients are grouped into (1) ICON-1 monotherapy, (2) Ranibizumab monotherapy, or (3) ICON-1 and Ranibizumab therapy treatment groups. In some embodiments the BCVA letter score is repeated at 0 month, 1 month, 2 months, 3 months, 4 months, 5 months, or 6 months. In some embodiments the CNV is classical CNV. In other embodiments, the CNV is occult. In one embodiment, the assessment BCVA letter score determinations occur as a last observation carried forward (LOCF) method.

In some embodiments, a patient gains greater than 5, 10, 15, 20, 25, 30, 35, or 40 letters in the BCVA letter score at 1 month, 2 months, 3 months, 4 months, 5 months, or 6 months after beginning treatment. In some embodiments, a patient gains greater than about 5, about 10, about 15, about 20, about 25, about 30, about 35, or about 40 letters in the BCVA letter score at 1 month, 2 months, 3 months, 4 months, 5 months, or 6 months after beginning treatment.

In one embodiment, the central subfield retinal thickness in the eye is determined in a patient or a population of patients wherein patients are grouped into (1) ICON-1 monotherapy, (2) Ranibizumab monotherapy, or (3) ICON-1 and Ranibizumab therapy treatment groups. In some embodiments the central subfield retinal thickness determination is repeated at 0 month, 1 month, 2 months, 3 months, 4 months, 5 months, or 6 months. In some embodiments the CNV is classical CNV. In other embodiments, the CNV is occult. In one embodiment, the central subfield retinal thickness determinations occur as a last observation carried forward (LOCF) method. In one embodiment, the central subfield retinal thickness determination is made utilizing sdOCT.

In some embodiments, a patient exhibits an increase or decrease in the central subfield retinal thickness by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment.

In some embodiments, a patient exhibits an increase or decrease in the central subfield retinal thickness by at least 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, the patient exhibits an increase or decrease in the central subfield retinal thickness of the tissues and/or regions of the eye presented herein is an increase or decrease of at least about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, or about 700 μm.

In one embodiment, the measure of thickness of the tissues and/or regions of the eye presented herein is an increase or decrease of at least 10 μm, 20 μm, 30 μm, 40 μm, 50 μm, 60 μm, 70 μm, 80 μm, 90 μm, 100 μm, 125 μm, 150 μm, 175 μm, 200 μm, 225 μm, 250 μm, 275 μm, 300 μm, 325 μm 350 μm, 375 μm, 400 μm 425 μm, 450 μm, 475 μm 500 μm, 525 μm, 550 μm, 575 μm, 600 μm, 625 μm, 650 μm, 675 μm, or 700 μm.

In one embodiment, a measure of the CNV area is taken in a patient or a population of patients wherein patients are grouped into (1) ICON-1 monotherapy, (2) Ranibizumab monotherapy, or (3) ICON-1 and Ranibizumab therapy treatment groups. In some embodiments the CNV area determination is repeated at 0 month, 1 month, 2 months, 3 months, 4 months, 5 months, or 6 months. In some embodiments the CNV is classical CNV. In other embodiments, the CNV is occult CNV. In one embodiment, the determinations of the CNV areas occur as a. last observation carried forward (LOCF) method.

In some embodiments, a patient exhibits a decrease in the CNV area by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment.

In some embodiments, a patient exhibits a decrease in the CNV area by at least at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, a measure of the area of leaking CNV is taken in a patient or a population of patients wherein patients are grouped into (1) ICON-1 monotherapy, (2) Ranibizumab monotherapy, or (3) ICON-1 and Ranibizumab therapy treatment groups. In some embodiments the area of CNV leaking is determination at 0 month, 1 month, 2 months, 3 months, 4 months, 5 months, or 6 months after beginning treatment. In some embodiments the CNV is classical CNV. In other embodiments, the CNV is occult CNV. In one embodiment, the measure of the area of leaking CNV occurs as a last observation carried forward (LOCF) method.

In some embodiments, a patient exhibits a decrease in the area of leaking CNV by at least 5%, 10%, 15%, 20%, 25%, 30%, 35%, 40%, 45%, 50%. 55%, 60%, 65%, 70?, 75%, 80%, 85%, 90%, 95%, or 100% at 1 month, 2 months, 3 months, 4 months, .5 months, or 6 months after beginning treatment. In some embodiments the CNV is classical CNV. In other embodiments, the CNV is occult.

In some embodiments, a patient exhibits a decrease in the area of leaking CNV by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, a measure of the volume of the sub-retinal fluid is taken in a patient or a population of patients wherein patients are grouped into (1) ICON-1 monotherapy, (2) Ranibizumab monotherapy, or (3) ICON-1 and Ranibizumab therapy treatment groups. In some embodiments the measure of the volume of the sub-retinal fluid is determined at 0 month, 1 month, 2 months, 3 months, 4 months, 5 months, or 6 months after beginning treatment. In some embodiments the CNV is classical CNV, In other embodiments, the CNV is occult CNV. In one embodiment, the measure of the measure of the volume of the sub-retinal fluid occurs as a last observation carried forward (LOCF) method.

In some embodiments, a patient exhibits a decrease or increase in the volume of the sub-retinal fluid by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment, In some embodiments the CNV is classical CNV. In other embodiments, the CNV is occult CNV.

In some embodiments, a patient exhibits a decrease or increase in the volume of the sub-retinal fluid by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, a measure of the thickness of the central subfield subretinal hyper-reflective material is taken in a patient or a population of patients wherein patients are grouped into (1) ICON-1 monotherapy, (2) Ranibizumab monotherapy, or (3) ICON-1 and 1Ranibizurnab therapy treatment groups. In some embodiments the measure of the thickness of the central subfield subretinal hyper-reflective material is determined at 0 month, 1 month, 2 months, 3 months, 4 months, 5 months, or 6 months after beginning treatment. In sonic embodiments the CNV is classical CNV. In other embodiments, the CNV is occult CNV. In one embodiment, the measure of the thickness of the central subfield subretinal hyper-reflective material occurs as a last observation carried forward (LOCF) method.

In some embodiments, a patient exhibits a decrease or increase in the thickness of the central subfield subretinal hyper-reflective material by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment. In some embodiments the CNV is classical CNV. In other embodiments, the CNV is occult CNV.

In some embodiments, a patient exhibits a decrease or increase in the thickness of the central subfield subretinal hyper-reflective material by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, a measure of the total subretinal hyper-reflective material volume is taken in a patient or a population of patients wherein patients are grouped into (1) ICON-1 monotherapy, (2) Ranibizumab monotherapy, or (3) ICON-1 and Ranibizumab therapy treatment groups. In some embodiments the measure of the total volume of the subretinal hyper-reflective material is determined at 0 month, 1 month, 2 months, 3 months, 4 months, 5 months, or 6 months after beginning treatment. In some embodiments, distinctions are made between subfoveal versus non-subfoveal. In some embodiments the CNV is classical CNV. In other embodiments, the CNV is occult CNV. In one embodiment, the measure of the total volume of the subretinal hyper-reflective material occurs as a last observation carried forward (LOCF) method.

In some embodiments, a patient exhibits a decrease or increase in the total volume of the subretinal hyper-reflective material by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment. In some embodiments the CNV is classical CNV. In other embodiments, the CNV is occult CNV. In some embodiments, distinctions are made between subfoveal versus non-subfoveal.

In some embodiments, a patient exhibits a decrease or increase in the total volume of the subretinal hyper-reflective material by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment. In some embodiments, distinctions are made between subfoveal versus non-subfoveal.

In one embodiment, the identification of the presence or absence is made for (1) intraretinal fluid, (2) subretinal fluid, (3) subretinal pigment epithelium fluid in a patient or a population of patients wherein patients are grouped into (1) ICON-1 monotherapy, (2) Ranibizumab monotherapy, or (3) ICON-1 and Ranibizumab therapy treatment groups. In some embodiments the determination of the presence or absence of fluid in said ocular locations is determined at 0 month, 1 month, 2 months, 3 months, 4 months, 5 months, or 6 months after beginning treatment. In some embodiments the CNV is classical CNV. In other embodiments, the CNV is occult CNV. In one embodiment, the determination of the presence or absence of fluid in said ocular locations occurs as a last observation carried forward (LOCF) method.

In some embodiments, a patient exhibits a presence or absence of (1) intraretinal fluid, (2) subretinal fluid, and/or (3) subretinal pigment epithelium fluid at 1 month, 2 months, 3 months, 4 months, 5 months, or 6 months after beginning treatment.

In some embodiments, a patient exhibits a presence or absence of (1) intraretinal fluid, (2) subretinal fluid, and/or (3) subretinal pigment epithelium fluid at 1 month, 2 months, 3 months, 4 months, 5 months, or 6 months after beginning treatment.

In one embodiment, the identification of the presence or absence of subfoveal or non-subfoveal cysts in a patient or a population of patients wherein patients are grouped into (1) ICON-1 monotherapy, (2) Ranibizumab monotherapy, or (3) ICON-1 and Ranibizumab therapy treatment groups. In some embodiments the determination of the presence or absence of subfoveal or non-subfoveal cysts is determined at 0 month, 1 month, 2 months, 3 months, 4 months, 5 months, or 6 months after beginning treatment. In some embodiments the CNV is classical CNV. In other embodiments, the CNV is occult CNV In one embodiment, the determination of the presence or absence of said cysts occurs as a last observation carried forward (LOCF) method.

In some embodiments, a patient exhibits a presence or absence of subfoveal or non-subfoveal cysts at 1 month, 2 months, 3 months, 4 months, 5 months, or 6 months after beginning treatment. In some embodiments, a patient exhibits a decrease in the presence of subfoveal or non-subfoveal cysts at 1 month, 2 months, 3 months, 4 months, 5 months, or 6 months after beginning treatment.

In some embodiments, a patient exhibits a presence or absence of subfoveal or non-subfoveal cysts at 1 month, 2 months, 3 months, 4 months, 5 months, or 6 months after beginning treatment. in some embodiments, a patient exhibits a decrease in the presence of subfoveal or non-subfoveal cysts at 1 month, 2 months, 3 months, 4 months, 5 months, or 6 months after beginning treatment.

In one embodiment, the identification of atrophy and/or fibrosis is made for the eye in a patient or a population of patients wherein patients are grouped into (1) ICON-1 monotherapy, (2) Ranibizumab monotherapy, or (3) ICON-1 and Ranibizumab therapy treatment groups. In some embodiments the identification of atrophy and/or fibrosis is determined at 0 month, 1 month, 2 months, 3 months, 4 months, 5 months, or 6 months after beginning treatment. In some embodiments the CNV is classical CNV. In other embodiments, the CNV is occult CNV. In one embodiment, the determination of the presence or atrophy and/or fibrosis occurs as a last observation carried forward (LOCF) method.

In some embodiments, a patient exhibits a decrease in the atrophy and/or fibrosis of the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, months, or 6 months after beginning treatment.

In some embodiments, a patient exhibits a decrease in the atrophy and/or fibrosis of the eye by at least about 5%, about 10%, about 15%, about 20%, about 25%, about 30%, about 35%, about 40%, about 45%, about 50%, 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, the total area of decreased autofluorescence is determined for the in a patient or a population of patients wherein patients are grouped into (1) ICON-1 monotherapy, (2) Ranibizumab monotherapy, or (3) ICON-1 and Ranibizumab therapy treatment groups. In some embodiments, the determination of the area of decreased autofluorescence is determined at 0 month, 1 month, 2 months, 3 months, 4 months, 5 months, or 6 months after beginning treatment. In some embodiments the CNV is classical CNV. In other embodiments, the CNV is occult CNV. In one embodiment, the determination of the total area of decreased autofluorescence occurs as a last observation carried forward (LOCF) method.

In some embodiments, a patient exhibits a decrease in the total area of decreased autofluorescence the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment.

In some embodiments, a patient exhibits a decrease in the total area of decreased autofluorescence the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment

In one embodiment, the total area of discontinuous autofluorescence in the eye is determined for a patient or a population of patients wherein patients are grouped into (1) ICON-1 monotherapy, (2) Ranibizumab monotherapy, or (3) ICON-1 and Ranibizumab therapy treatment groups. In some embodiments, the determination of the total area of discontinuous autofluorescence is determined at 0 month, 1 month, 2 months, 3 months, 4 months, 5 months, or 6 months after beginning treatment. In some embodiments the CNV is classical CNV. In other embodiments, the CNV is occult CNV. In one embodiment, the determination of the total area of discontinuous autofluorescence occurs as a. last observation carried forward (LOCF) method.

In some embodiments, a patient exhibits a decrease in the total area of discontinuous autofluorescence the eye by at least 5%, 10%, 15%, 20%, 25%, 30%, 35%, 40%, 45%, 50%, 55%, 60%. 65%, 70%, 75%, 80%, 85%, 90%, 95%, or 100% at 1 month, 2 months, 3 months, 4 months, 5 months, or 6 months after beginning treatment.

In some embodiments, a patient exhibits a decrease in the total area of discontinuous autofluorescence the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, a measurement of the volume of the central subfield pigment epithelium detachment is determined for in a patient or a population of patients wherein patients are grouped into (1) ICON-1 monotherapy, (2) Ranibizumab monotherapy, or (3) ICON-1 and Ranibizumab therapy treatment groups. In some embodiments, the determination of the volume of the central subfield pigment epithelium detachment is determined at 0 month, 1 month, 2 months. 3 months. 4 months, 5 months, or 6 months after beginning treatment. In some embodiments the CNV is classical CNV. In other embodiments, the CNV is occult CNV. In one embodiment, the volume of the central subfield pigment epithelium detachment occurs as a last observation carried forward (LOCF) method.

In some embodiments, a patient exhibits a decrease in the volume of the central subfield pigment epithelium detachment by at least 5%, 10%, 15%, 20%, 25%, 30%, 35%, 40%, 45%, 50%, 55%, 60%, 65%, 70%, 80%, 85%, 90%, 95%, or 100% at 1 month, 2 months, 3 months, 4 months, 5 months, or 6 months after beginning treatment.

In some embodiments,a patient exhibits a decrease in the volume of the central subfield pigment epithelium detachment by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

In one embodiment, a determination of the integrity of the (1) outer nuclear layer, (2) external limiting membrane, (3) ellipsoid zone, and (4) subfoveal retinal pigment epithelium of an eye is made for a patient or a population of patients wherein patients are grouped into (1) ICON-1 monotherapy, (2) Ranibizumab monotherapy, or (3) ICON-1 and Ranibizumab therapy treatment groups. In some embodiments, the determination of the integrity of (1)-(4) is determined at 0 month, 1 month, 2 months, 3 months, 4 months, 5 months, or 6 months after beginning treatment. In some embodiments the CNV is classical CNV. In other embodiments, the CNV is occult CNV. In one embodiment, the determination of the integrity of (10-(4) occurs as a last observation carried forward (LOCF) method.

1001431 In some embodiments, a patient exhibits an increase in the integrity of the (1) outer nuclear layer, (2) external limiting membrane, (3) ellipsoid zone, and (4) subfoveal retinal pigment epithelium of the eye by at least 5%, 10%, 15%, 20%, 25%, 30%, 35%, 40%, 45%, 50%, 55%, 60%, 65%, 70%, 75%, 80%, 85%, 90%, 95%, or 100% at 1 month, 2 months, 3 months, 4 months, 5 months, or 6 months after beginning treatment.

In some embodiments, a patient exhibits an increase in the integrity of the (1) outer nuclear layer, (2) external limiting membrane, (3) ellipsoid zone, and (4) subfoveal retinal pigment epithelium of the eye by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% at 1 month, 2 months, 3 months, 4 months, 5 months, or 6 months after beginning treatment.

### EXAMPLES

The present invention is further illustrated by reference to the following Examples. However, it should be noted that these Examples, like the embodiments described above, are illustrative and are not to be construed as restricting the scope of the invention in any way.

### Example 1

### Evaluation of hI-con1 In in vitro Thrombin Generation Assays

The effect of hI-con1 (SEQ ID NO:2) in a thrombin generation assay in plasma was tested. Specifically, the effect of hI-con1 on thrombin generation in plasma in a tissue factor initiated reaction using a thrombogram (CAT-like) assay (Hemker et al. 2002. Pathophysiol. Haemost. Thromb. 32, pp. 249-253; Mann et al. 2007. J. Thromb Haemost. 5, pp. 2055-2061, each incorporated by reference herein in its entirety for all purposes) was evaluated. For the CAT-like assays, multidonor human citrate plasma from healthy individuals, human FVII-deficient plasma and normal rabbit citrate plasma were used. Thrombin (also referred to as Factor IIa, or activated blood coagulation factor II) generation was initiated either with human relipidated TF (in human plasma) or with rabbit relipidated TF (in rabbit plasma).

hI-con1 was maintained frozen at −70° C. until use. Each sample included 3.0 mg hI-con1/mL in formulation buffer (15 mM HEPES, 150 mM NaCl, 5 mM CaCl₂, 25 mM Arginine, 0.01% Tween 80, pH 7.4).

Human plasma FVIIa in 50% glycerol was purchased from Haematologic Technologies, Inc., 57 River Road, Essex Junction, VT 05452. It was stored at −20° C. until use. Before use, it was diluted to 10 nM in the formulation buffer (15 mM HEPES, 150 mM NaCl, 5 mM CaCl2, 25 mM Arginine, 0.01% Tween 80, pH 7.4).

Spectrozyme FXa (# 222), lipidated recombinant human TF reagent (Catalog # 4500L) and lipidated recombinant rabbit TF were purchased from American Diagnostica, Inc. (Stamford, Conn.), pooled normal human plasma (Lot # IR 11-020711) and rabbit plasma (Lot # 26731) were purchased from Innovative Research Novi, Mich. 48377), congenital FVII-deficient plasma (Catalog # 0700) was purchased from George King Bio-Medical, Inc. (Overland Park, Kans.) and human factor X (hFX) (# HCX-0050) and Phe-Pro-Arg-chloromethylketone (FPRck; Catalog # FPRCK-01), corn trypsin inhibitor (CTI; Catalog # CTI-01) were purchased from Haematologic Technologies, Inc (Essex Junction, Vt., USA). Fluorogenic substrate Z-Gly-Gly-Arg-AMC·HCl was purchased from Bachem (Torrance, Calif.) and ethylenediaminetetraacetic acid disodium salt dihydrate (EDTA; # E5134), NaCl (# S7653) and HEPES (# H3375) were purchased from Sigma (St. Louis, Mo.). FIBS buffer, pH 7.4 contained 150 mM NaCl, 2 mM CaCh and 20 mM HEPES.

Active site inhibited FVIIa. (FVIIai) was produced in house. 1, 2-Dioleolyl-sn-Glycero-3-Phospho-L-Serine (PS; # 840035) and 1, 2-DioleoyJ-sn-Glycero-3-Phosphocholine (PC; # 850375) were purchased from Avanti Polar Lipids, Inc. (Alabaster, Ala., USA). Phospholipid vesicles (PCPS) composed of 25% PS and 75% PC were prepared as described in Higgins and Mann 1983, incorporated by reference herein in its entirety for all purposes.

### Extrinsic FXase

Lipidated recombinant human TF (0.1 nM) was incubated with either 5 nM plasma FVIIa or 5 nM hI-con1 or mixture of both (each at 5 nM) and 100 μM PCPS for 10 min at 37° C. FX (4 μM) was added and at selected time points (0-5 min.) 10 μL aliquots of the reaction mixture were quenched into 170 μL HBS-0.1% PEG-20 mM EDTA. Twenty μL of Spectrozyme FXa (0.2 mM) was added and the rate of substrate hydrolysis was measured as an increase in absorbance at 405 nm (mOD/min).

### Thrombin Generation (CAT-Like) Assay

Corn trypsin inhibitor (CTI) at a final 0.1 mg/mL concentration was added to citrate plasma and 80 μL of this plasma was transferred into ImmuIon® 96-well plate (Thermo Electron Co., Waltham Mass.). When desired, hI-con1, plasma FVIIa and FVIIai were added at selected concentrations. Twenty μL of 5 pM TF and 20 μM PCPS mixture (both concentrations final) were added to CTI-plasma and incubated for 3 min. Thrombin generation was initiated by the addition of 20 μL of 2.5 mM ZGly-Gly-ArgAMC.HCl in HBS containing 0.1 M CaCl₂. Final concentration of substrate was 416 μM and that of CaCl₂ was 15 mM. Thrombin generation curves were generated using Thrombinoscope BY software.

### Results

Comparison of hI-con1 with Plasma FVIIa in the Extrinsic FXase

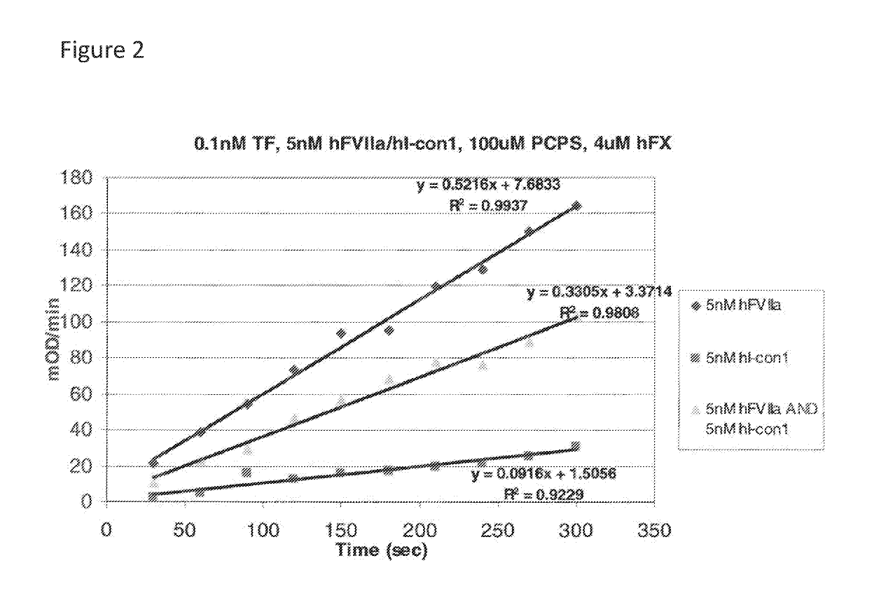

FXa-generating efficiency of two forms of FVIIa and of their mixture was determined in a chromogenic assay. hI-con1 was less active than plasma FVIIa. Activity of hI-con1 was 18% of that observed for plasma FVIIa. When both proteins were added at equimolar (5 nM) concentration, the rate of FXa generation in the middle between the rates observed for individual proteins, indicating that hI-con1 competes with plasma FVIIa for the limited amount of TF (FIG. 2). These data also suggest that hI-con1 has similar affinity for TF as plasma FVIIa.

### Thrombin Generation in Normal Human Plasma: the Effect of FVIIai

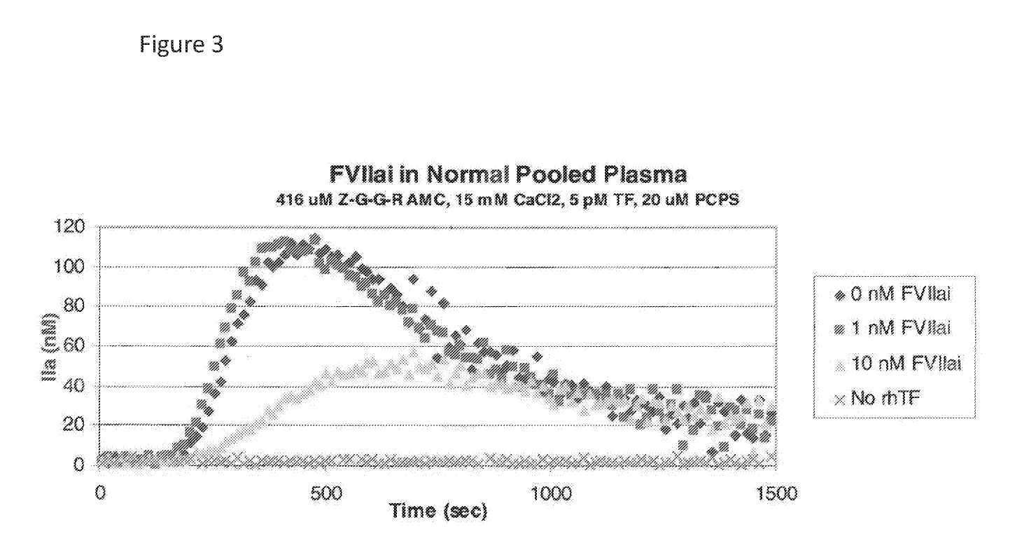

It was hypothesized that due to the low activity of the hI-con1 tissue factor (TF) complex in the extrinsic FXase, hI-con1 could act as an inhibitor by binding TF into an inefficient complex and preventing formation of an efficient complex between plasma FVIIa and TF. To test this hypothesis, the effect of a known inhibitor of coagulation, i.e., active site inhibited FVIIa (Kjalke et al. 1997), on thrombin generation in normal human plasma was evaluated. FVIIai at 1 nM concentration had no effect on thrombin generation initiated with lipidated human TF (FIG. 3). However at 10 nM, FVIIai prolonged the lag phase of thrombin generation and significantly suppressed both the maximum rate of thrombin generation and the maximum levels of thrombin produced. No thrombin generation was observed in the absence of TF.

Thrombin Generation in Normal Human Plasma: the Effect of hI-con1

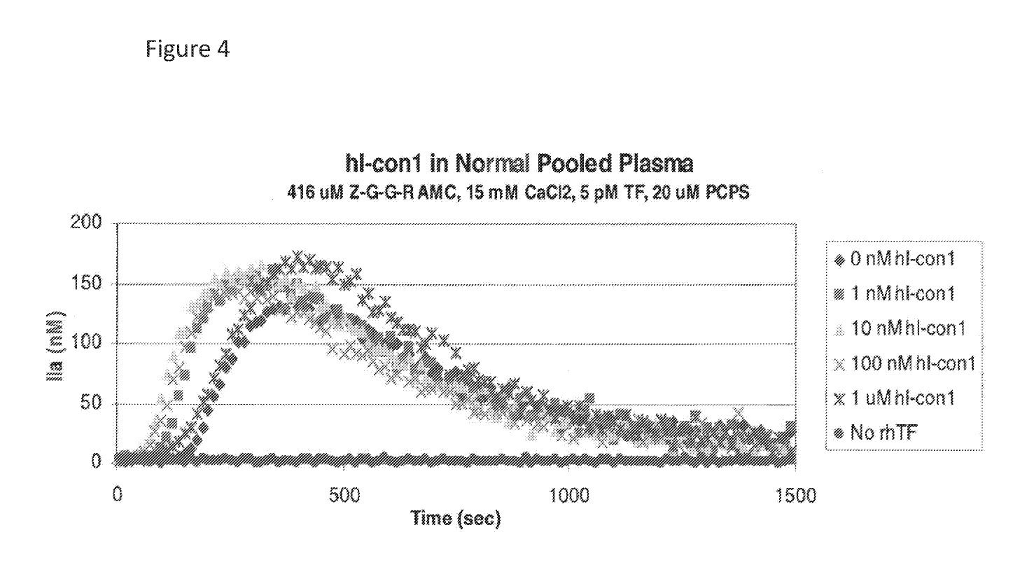

hI-con1 was titrated into normal human plasma initiated with TF to generate thrombin. Varying concentrations of hI-con1 was used, however even at extremely high hI-con1 concentrations (1 μM), no inhibition of thrombin generation was observed (FIG. 4).

### Thrombin Generation min Congenital FVII-deficient Human Plasma

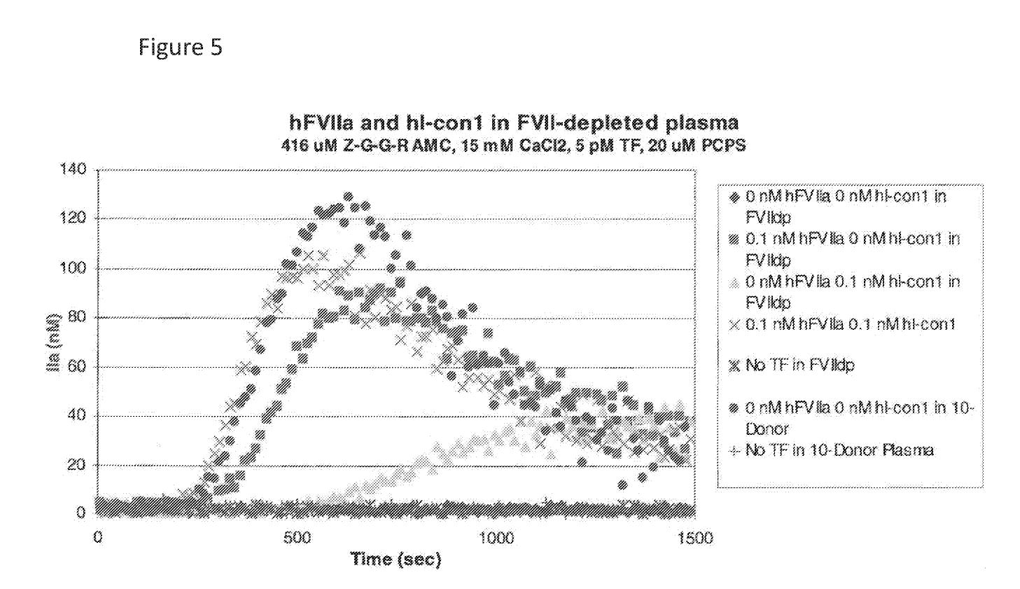

No thrombin generation was observed upon the addition of lipidated human TF to congenital FVII-deficient plasma, indicating that there no detectable functional FVIIa in that plasma (FIG. 5). An addition of 0.1 nM plasma FVIIa together with TF produced thrombin generation profile slightly lower than that observed in normal human plasma. An addition of 0.1 nM hI-con1 alone in the presence of TF led to the initiation of thrombin generation, however the process was significantly delayed and suppressed (FIG. 5). This result was consistent with the observation of low hI-con1 activity in the extrinsic FXase. The addition of both plasma FVIIa and hI-con1 at equimolar concentrations (0.1 nM) did not impair thrombin generation initiated with plasma FVIIa alone.

### Thrombin Generation in Normal Rabbit Plasma

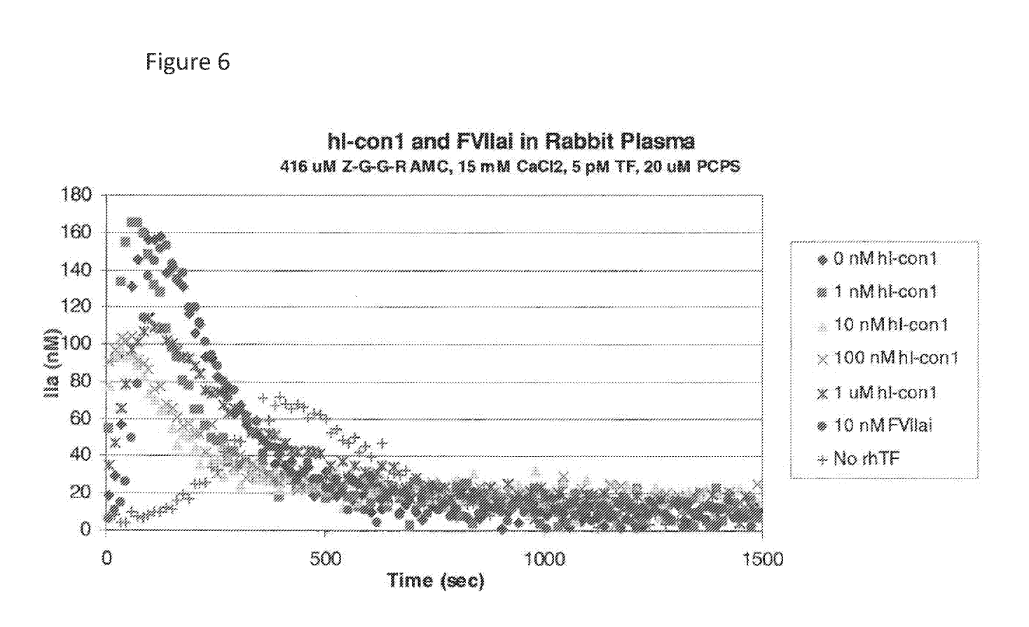

Thrombin generation in rabbit plasma was initiated with lipidated rabbit TF. The addition of 1 nM hI-con1 to this plasma had no pronounced effect on thrombin generation (FIG. 6). Similarly, no pronounced effect was observed when 10 nM FVIIai was added. At higher hI-con1 concentrations (10-1000 nM) some suppression in thrombin generation was observed. However the control experiment with no TF added led to thrombin generation, suggesting an endogenous presence of TF.

### Thrombin Generation in Centrifuged Rabbit Plasma

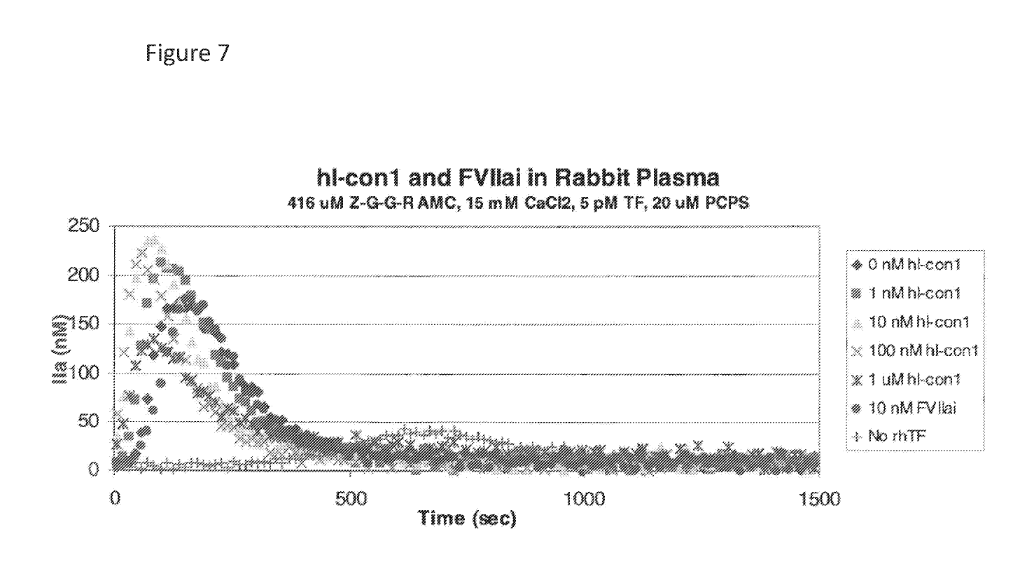

After centrifugation of rabbit plasma, an endogenous thrombin generating activity did not disappear completely, but was significantly decreased (FIG. 7). No suppression in TF-triggered thrombin generation was observed when 10 nM FVIIai was added. Similarly, no suppression was observed when 1-100 nM hI-con1 was added and only a limited decrease in thrombin generation was observed when high concentration (1 μM) hI-con1 was added (FIG. 7). These data indicate that at physiologically-relevant concentrations hI-con1 does not compete for rabbit TF with rabbit FVIIa.

### Conclusions

hI-con1 does not compete with plasma FVIIa for TF in the citrate plasma environment. hI-con1 has no pronounced (if any) effect on thrombin generation either in human plasma initiated with human TF or in rabbit plasma initiated with rabbit TF. It is not likely that hI-con1 would cause bleeding or thrombotic complications.

### Example 2

### Effects of Treatment With hI-con1 on Choroidal Neovascularization in the Pig

In this study, hI-con1 activity in a porcine wet AMD model (Kiilgaard et al., 2005. Acta. Ophthalmol. Scand. 83, pp. 697-704, incorporated by reference herein in its entirety for all purposes) and the optimal dose for the activity was examined. Additionally, the safety of hI-con1 when administered by intravitreal injection was determined.

In this study intravitreal injection of hI-con1 was demonstrated to result in the destruction of established laser-induced CNV in this porcine model. The injections of hI-con1 were well tolerated and the effects were dose-related, with and ED₅₀ o f13.5 μg/dose. A major breakdown product of hI-con1 (100kDa) was tested and was also well-tolerated and effective with an ED₅₀ of 16.2 μg/dose.

### Test Articles

hI-con1

hI-con1 was provided by Laureate Pharma Inc., 201 E. College Ave, Princeton, N.J., 08540. hI-con1 was maintained frozen at −70° C. until use: Lot PURIC1 080402 (SEC Fr 10-14), two vials each containing 200 μL at 2.0 mg/mL, 1.0 mg/mL, 0.5 mg/mL and 0.25 mg/mL in formulation buffer (15 mM HEPES, 150 mM NaCl, 5 mM CaCl₂, 25 mM Arginine, 0.01% Tween 80, pH 7.4).

100 kD Fragment of hI-con1

The following samples of the 100 kD fragment of hI-con1 were provided by Laureate Pharma Inc. 201 E. College Ave, Princeton, N.J., 08540. The fragment was maintained frozen at −70° C. until use: Lot PURIC1 080402 (SEC Fr 15), two vials each containing 200 μL at 2.0 mg/mL, 1.0 mg/ mL, 0.5 mg/mL and 0.25 mg/mL in formulation buffer (15 mM HEPES, 150 mM NaCl, 5 mM CaCl₂, 25 mM Arginine, 0.01% Tween 80, pH 7.4).

### Control Article

The formulation buffer (15 mM HEPES, 150 mM NaCl, 5 mM CaCl₂, 25 mM Arginine, 0.01% Tween 80, pH 7.4) was used as the vehicle control.

### Test Animals

Two studies were conducted, each with groups of five (one group per test article) Yucatan miniature pigs (Sus scrofa), 10-12 weeks old, each weighting approximately 20 kilograms were bought from Professional Veterinary Research (Brownstown, Ind., USA).

### Husbandry

Each pig was maintained in a separate cage within a communal environment that housed four pigs. The lighting was computer controlled and set for a 6 am to 6 pm cycle. The temperature average was 70-72° F., with a variation of +/−1 degree. Humidity was kept between 30 and 70%, with average humidity equal to 33%. The animals were evaluated by the large animal husbandry supervisor and a licensed veterinary technician on arrival and a licensed veterinary technician once weekly until they were euthanized. A veterinarian evaluated the animals to determine if there were any abnormalities or concerns. The animals were quarantined for about 1 week prior to experiments.

### Feed and Water

Daily feed and water were provided to the miniature pigs. They were bedded on hay that served as a feed supplement. The feed was Purina # 5084, Laboratory Porcine Grower Diet, Manufactured by Purina Mills, LLC, 555 Maryville University Drive, Suite 500, St. Louis, Mo. 63141, and fed at 2% body weight per day. The water was 0.5 micron filtered tap water. It was not routinely analyzed for contaminants except by the water company and reports are reviewed annually.

### Justification of Species

hI-con1 has limited cross-species activity and the pig is one of the few laboratory animal species in which it is active. The vitreous cavity of the pig is approximately 3 mL, allowing intravitreal injection of reasonable volumes of test article. The pig eye has retinal vascular similarities to humans in addition to several cone-dominant regions of the retina that are similar to the human macula.

### Methods

### Laser-Induced Choroidal Neovascularization

Under general anesthesia, the pupils of the animals were dilated with 1% tropicamide and 2.5% phenylephrine. An indirect ophthalmoscope with a double-frequency YAG laser (532 nm) was used to deliver 74 spots per eye using a 2.2. D lens and the following laser parameters: laser power 1000-1500 mW, duration 0.1 seconds, and repetition rate 500 msec. The laser treatment was designed to yield a microrupture of the Bruch's membrane, generating CNV at 60-70% of the laser spots within two weeks (Bora et al., 2003, incorporated by reference herein in its entirety for all purposes).

### Study Design

The study design is summarized in Table 3 below.

TABLE 3
Study design.

Dose injected (μg)
in each eye

| | | hI-con1 100 kD |
|---|---|---|
| Pig Number | hI-con1 | fragment |
| 1 | 0 | 0 |
| 2 | 25 | 25 |
| 3 | 50 | 50 |
| 4 | 100 | 100 |
| 5 | 200 | 200 |

Choroidal neovascularization was induced on Day 0 in both eyes of two groups of 5 pigs. On Day 10, 100 μL of solutions of hI-con1 (Study 1) or its 100 kD fragment (Study 2) at 0.25, 0.5, 1.0 or 2.0 mg/mL were administered by intravitreal injection into both eyes of the pigs as shown in Table 3. On Day 10, 100 μL of formulation buffer was administered by intravitreal injection into both eyes of the control pigs.

### Test and Control Articles Administration

The animals were anesthetized with a mixture of ketamine hydrochloride (40 mg/kg) and xylazine hydrochloride (10 mg/kg). Injections were administered using a strict sterile technique, which involved scrubbing the lids with a 5% povidone-iodine solution and covering the field with a sterile eye drape. A sterile lid speculum was used to maintain exposure of the injection site. All injections were performed 2 mm from the limbus through the pars plana, using a 30-gauge needle on a 1 mL tuberculin syringe. After injection, a drop of 2% cyclopentolate and antibiotic ointment was placed in the eye. The animals were examined daily for signs of conjunctival injection, increased intraocular pressure, anterior uveitis vitritis, or endophthalmitis, and were sacrificed on Day 14.

### Terminal Procedures

On Day 14, the pigs were anesthetized with an 8:1 mixture of ketamine and xylazine and perfused through the ear vein with 10 mL PBS containing 3 mg/mL fluorescein-labeled dextran with an average molecular weight 2×10⁶ (Sigma, St. Louis, Mo., USA). The eyes were enucleated and four stab incisions were made at the pars plana followed by fixation in 4% paraformaldehyde for 12 hours at 4° C. The cornea and the lens were removed, and the neurosensory retina was dissected from the eyecup and four radial cuts were made from the edge of the eyecup to the equator. The choroid-retinal pigment epithelium (RPE) complex was separated from the sclera and flatmounted on a glass slide in Aquamount with the inner surface (RPE) facing upwards. Flat mounts were stained with a monoclonal antibody against elastin (Sigma) and a Cy3-conjugated secondary antibody (Sigma) and examined with a confocal microscope (Zeiss LSM510, Thornwood, N.Y., USA). The vasculature, filled with dextranconjugated fluorescein, stained green and the elastin in the Bruch's membrane stained red. The level of the Bruch's membrane was determined by confocal microscopy using the intense red signal within a series of z-stack images collected at and around the laser spot. The presence of CNV was indicated by the branching linear green signals above the plane of Bruch's membrane. Absence of CNV was defined under very stringent criteria as the total absence of green fluorescence in the vessels in the spot (see Tezel et al., 2007. Ocular Immunol Inflamm 15, pp. 3-10.

### Statistical Analysis

The percentage of laser spots with CNV at different doses of hI-con1 or its 100 kD fragment was compared pair wise by a chi-square test. The results were plotted against the hI-con1 dose to derive the beat-fit curve, which was used to calculate the dose of hI-con1 that reduces the fraction of laser spots with CNV by 50% (ED50). A confidence level of p <0.05 was considered to be statistically significant.

### Results

Effects of Intravitreal Treatment with hI-con1 on CNV

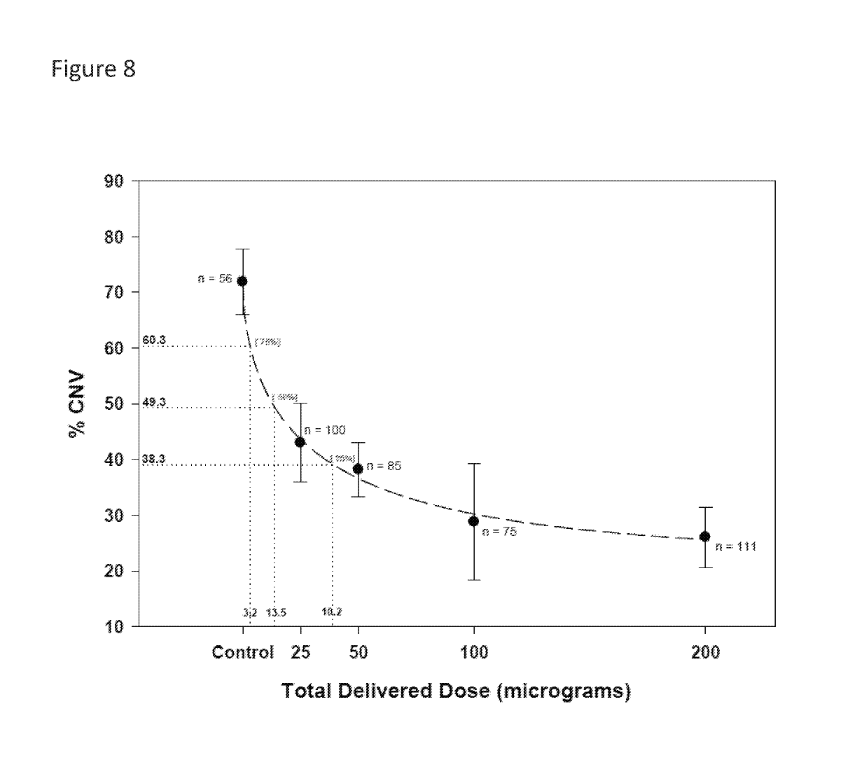

Choroidal neovascularization developed in 71.9±5.8% of the laser spots in control eyes. A single intravitreal injection of hI-con1 on Day 10 in pig eyes (n=2 at each dose) significantly reduced subretinal CNV on Day 14 at all doses tested, i.e., 25-200 μg, Table 4; FIG. 8). The inhibitory effect of hI-con1 fit well to a 5-parameter Sigmoidal Weibull curve. The dose causing a 50% decrease in the yield of CNV (ED50) was 13.5 μg.

TABLE 4
Effects of intravitreal treatment with hI-con1 on CNV
incidence in laser-induced CNV pig model.

| | | | | % spots with | |
|---|---|---|---|---|---|
| | Laser Spots | | | CNV | |
| Pig | Examined | | hI-con1 dose | (Average ± | P value (vs. |
| Number | Left | Right | (μg) | SD) | control) |
| 1 | 25 | 31 | 0 | 71.9 ± 5.8 | |
| 2 | 50 | 50 | 25 | 43.0 ± 7.1 | 0.001 |
| 3 | 36 | 49 | 50 | 38.2 ± 4.9 | <0.001 |
| 4 | 47 | 28 | 100 | 28.8 ± 10.4 | <0.001 |
| 5 | 54 | 57 | 200 | 26.0 ± 5.4 | <0.001 |

Effects of Intravitreal Treatment with 100 kD fragment of hI-con1 on CNV

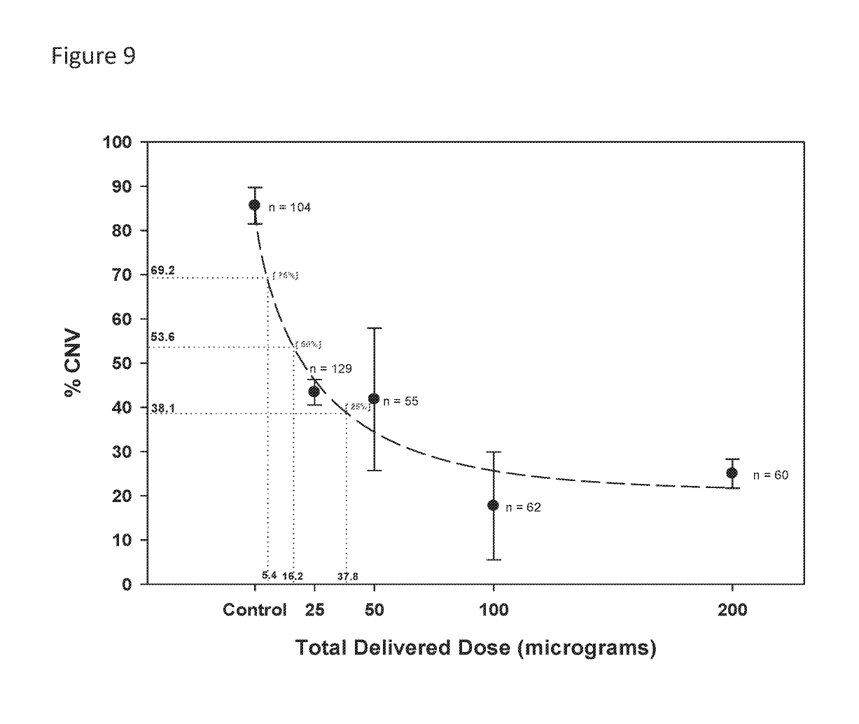

Choroidal neovascularization developed in 85.6±4.1% of the laser spots in control eyes. A single intravitreal injection of the 100 kDa fragment of hI-con1 on Day 10 in pig eyes (n=2 at each dose) significantly reduced subretinal CNV on Day 14 at all doses tested, i.e., 25-200 μg, Table 4, FIG. 9). The inhibitory effect of hI-con1 fit well to a 5-parameter Sigmoidal Weibull curve. The dose causing a 50% decrease in the yield of CNV (ED50) was 16.2 μg.

TABLE 5
Effects of intravitreal treatment with 100 kD fragment of
hI-con1 on CNV incidence in laser-induced CNV pig model.

| | | | | % spots with | |
|---|---|---|---|---|---|
| | Laser Spots | | | CNV | |
| Pig | Examined | | hI-con1 dose | (Average ± | P value (vs. |
| Number | Left | Right | (μg) | SD) | control) |
| 1 | 52 | 52 | 0 | 85.6 ± 4.1 | |
| 2 | 73 | 56 | 25 | 43.4 ± 2.9 | <0.001 |
| 3 | 26 | 29 | 50 | 41.8 ± 16.1 | <0.001 |
| 4 | 30 | 32 | 100 | 17.7 ± 12.2 | <0.001 |
| 5 | 14 | 46 | 200 | 25.0 ± 3.3 | <0.001 |

Intravitreal injection of hI-con1 and its 100 kD fragment at doses from 25-200 μg caused significant regression of pre-existing laser-induced CNV 4 days after the injections were administered. The response of the lesions to the injections was clearly dose-related with ED5o doses of 13.5 and 16.2 μg, respectively. These results indicate that the specific activity of the 100 kD fragment of hI-con1 is similar to that of the intact molecule. Doses greater than 100 μg had very little additional decrease in CNV; thus, the efficacious dose in this model is ≤100 μg.

### Example 3

### Tissue Cross-Reactivity Study of hI-con1 With Normal Human Tissues

In this study, the binding of hI-con1 to normal human tissues was assessed using standard immunohistochemistry (IHC) techniques, in a standard tissue cross-reactivity (TCR) study. The study was performed utilizing a single batch of biotinylated hI-con1 for IHC staining of normal, as well as positive and negative control human tissues. A positive staining result is indicative of potential toxicities associated with administration of hI-con1 to humans in vivo.

In this model, tissue staining was observed only in the positive control colon carcinoma tumor. All other normal human tissues showed no immunoreactivity. These findings indicate that hI-con1 binding is specific to abnormal tissue, with no binding to normal tissues observed.

### Example 4

### Evaluation of the Binding of hI-con1 to Lipidated Tissue Factors

To allow for cross species comparison, a Biacore study of the kinetics of binding of hI-con1 and hFVIIa to human lapidated TF (hTF) and rabbit lipidated TF (rTF) was conducted.

As described in detail below, hI-con1 and hFVIIa both bound with high and approximately equal affinity to lapidated

### Materials and Methods

Lipidated rabbit tissue factor (rTF; Product # 4520L; Lot # 051017) purchased from American Diagnostica. Lipidated human tissue factor (hTF; Lot FIL105HO1) supplied by Marin Biological Laboratories, 378 Bel Marin Keys, Novato, Calif. 94949.

hI-con1; 1 ml; 100 μg/ml; mMW 157 kDa

Human FVIIa; Lot # A09050525 (Fitzgerald); 1.01 mg/ml; 40 μL/vial; MST 50 kDa

### Equipment: Biacore 3000; CM5 Sensor Chip

The GE procedure for proteoliposome immobilization (amine coupling) protocol was used to coat PS/PC/rTF on flow cell 2, and PS/PC/hTF on flow cell 3. Flow cells were equilibrated with running buffer (15 mM Hepes, 150 mM NaCl, 5 mM CaCl2, 25 mM Arginine, 0.01% Tween 80, pH7.4) at a flow rate of 5 μL/min. Kinetic analyses were performed at 37° C. by flowing consecutively increasing concentrations of each analyte (0-10 nM) in the running buffer (15 mM HEPES, 150 mM NaCl, 5 mM CaCl2, 25 mM Arginine, 0.01% Tween 80, pH 7.4) over the sensor chip for 5 min followed by a 10 min dissociation period at a flow rate of 30 μL/min in parallel.

Analyte binding to the lipidated TF was determined by subtracting the RU values noted in the reference flow cell 1 from flow cell 2 and 3. Binding of analytes to the TF_{S} was monitored in real time to obtain on (ka) and off (kd) rates. The equilibrium dissociation constant (KD) was calculated from the observed ka and kd.

The chips were regenerated with 3 min pulses of 10 mM EDTA in HEPES buffer (20 mHEPES, 150 mM NaCl, pH 7.4).

Capture of rTF to the chip-Flow cell 2 was coated with rabbit TF (>Resonance Units [RU] 10,000) by amine coupling. Flow cell 3 was coated with human TF (>RU 8,000) by amine coupling.

### Determination of the Amount of Test Ligands (RL) to be Captured on the Chip

In this experiment, the desired level of R_{Max} for the measurement of ligand-analyte interaction was based on the value determined by a previous experiment where rTF captured at 10,000 Resonance Units (“RU”) gave binding of hI-con1 with R_{Max} of 15 RU and hTF captured at 8,000 RU gave binding of hI-con1 with R_{Max} of 10 RU. The amount of the analyte to be captured on the chip depended on the molecular weights of the interacting proteins. It is determined by the following formula:

R_{Max}=MW_{A}/MW_{L}·R_{L}

MW_{A} is the molecular weight of the analyte (157 kDa for hI-con1, 50 kDa for hFVIIa, and 150 kDa for IgG1).

MW_{L} is the molecular weight of the ligand, in this assay it is expected to be very large (multiple of 35 kDa).

### Flow Rate of the Antibody Solution

The flow rate used for capturing the ligand was 10 μL/min.

For kinetics analysis, the flow rate of 30 μL/min. was used.

### Kinetic Analysis

Based on the saturation concentration of the analyte, binding analysis was performed using saturating analyte concentrations of 0-500 nM for rabbit TF and 0-50 nM for human TF. Chi squared (χ²) analysis was carried out between the actual sensorgram and the calculated on- and off-rates to determine the accuracy of the analysis.

χ² value up to 2 is considered significant (accurate) and below 1 is highly significant (highly accurate).

### Results

The Biacore assay results are provided in Table 6 below.

TABLE 6
Biacore assay results

| | | Ka | Dk | Rmax | Conc. of | KA | K_{D} | Chi squared |
|---|---|---|---|---|---|---|---|---|
| Ligand | Analyte | (1/Ms) | (1/s) | (RU) | analyte | (1/M) | (M) | (χ²⁾ |
| Rabbit TF | hI-con1 | 5.6 × 10⁴ | 3.0 × 10⁻³ | 4.92 | 0-500 nM | 1.9 × 10⁷ | 5.3 × 10⁻⁸ | 5.6 × 104 |
| Rabbit TF | hFVIIa | 3.4 × 10⁴ | 1.4 × 10⁻³ | 4.9 | 0-500 nM | 2.5 × 10⁷ | 4.0 × 10⁻⁸ | 0.06 |
| Human TF | hI-con1 | 6.2 × 10⁵ | 5.5 × 10⁻⁴ | 71.5 | 0-50 nM | 1.1 × 10⁹ | 8.9 × 10⁻¹⁰ | 3.34 |
| Human TF | hFVIIa | 5.1 × 10⁵ | 9.3 × 10⁻⁴ | 47 | 0-20 nM | 5.5 × 10⁸ | 1.8 × 10⁻⁹ | 0.13 |

As shown in Table 7 below, hI-con1 and hFVIIa both bound with high, and approximately equal, affinity to lipidated hTF. Both ligands also bound to lipidated rTF with approximately 10-fold lower affinities.

TABLE 7
Relative affinities for the binding of hI-con1 and
hFVIIa to hTF and rTF

Dissociation Constant (K_{D})
Analyte

| Ligand | hI-con1 | hFVIIa |
|---|---|---|
| Human Tissue Factor | 8.92 × 10⁻¹⁰M | 1.81 × 10⁻⁹M |
| Rabbit Tissue Factor | 5.32 × 10⁻⁸M | 3.95 × 10⁻⁸M |

### Example 5

### Randomized, Double-Masked, Multicenter, Active-Controlled Study Evaluating hI-con1 in Patients With CNV Secondary to Age-Related Macular Degeneration

In this study, the safety of intravitreal injections of hI-con1, administered as monotherapy or in combination with ranibizumab (LUCENTIS) compared to ranibizumab monotherapy in patients with choroidal neovascularization (CNV) secondary to age-related macular degeneration (AMD) is assessed.

Additionally, the biological activity and pharmacodynamics effect of hI-con1, administered as monotherapy or in combination with ranibizumab (LUCENTIS) compared to ranibizumab monotherapy is assessed.

The study presented in this example is a randomized, double-masked, multicenter, active-controlled study. Patients enrolled in this study are naive to treatment for CNV. Patients are randomly assigned to one of the following three treatment arms in the selected study eye in a 1:1:1 ratio:
hI-con1 monotherapy (0.3 mg)+sham injection
ranibizumab monotherapy (0.5 mg)+sham injection
hI-con1 (0.3 mg) ranibizumab (0.5 mg) combination therapy

Randomization is stratified by best-corrected visual acuity (BCVA) letter score in the study eye at baseline (≤54 letters versus≥55 letters) and by study site.

Patients receive up to two intravitreal injections at each injection visit. In order to maintain the study mask among the treatment arms, a sham injection is employed in patients receiving monotherapy.

Patients are administered intravitreal injections in the study eye once every four weeks at months 0, 1 and 2. As of Month 3 (at Months 3, 4 and 5) patients are retreated according to their assigned treatment arm, based on their individual observed treatment response. The masked Investigator uses the following retreatment criteria (based on the category of individual patient response) to determine if treatment is required at these visits:
Loss of≥5 letters of BCVA due to AMD compared to the previous scheduled visit.
Independent of BCVA change, any anatomical evidenced of increased CNV activity (e.g., new or increased fluid and/or leakage, hemorrhage) compared to the previous scheduled visit.
No BCVA change compared to Baseline (Visit 2), but there is anatomical evidence of persistent CNV activity (e.g., same persistent fluid and CST compared to Baseline.

Rescue treatment with 0.5 mg of ranibizumab is administered to the study eye as an add-on therapy at any time during the 6-month treatment and follow-up period if either of the following conditions occur:
Loss of≥15 letters of BCVA due to AMD compared to Baseline (Visit 2).
Loss of≥10 letters from baseline (Visit 2) of BCVA due to AMD that is confirmed at two consecutive visits. Patients with a loss of ≥10 letters compared to baseline are requested to return within 7 days or as soon as possible for additional follow up at an unscheduled visit.

The masked physician will make the determination if rescue treatment is needed according to the above criteria.

If rescue treatment is administered to the study eye during a scheduled injection visit, to ensure that the study masking is maintained, the unmasked physician administers rescue treatment and the patient's scheduled study treatment/re-treatment is as follows.
hI-con1 monotherapy arm: hI-con1 (0.3 mg)+rescue therapy (0.5 mg ranibizumab).
ranibizumab monotherapy arm: ranibizumab (0.5 mg)+sham injection.
combination therapy: hI-con1 (0.3 mg)+ranibizumab (0.5 mg).

If rescue treatment is administered to the study eye at an unscheduled visit, the unmasked physician administers rescue treatment as requested.

If rescue treatment is administered to the study eye, the patient continues with the study visit schedule for the next visit in accordance with the protocol and continues receiving study treatment according to the assigned randomization arm.

Safety is evaluated by tracking of adverse events, clinical laboratory tests (serum chemistry, hematology and coagulation), vital signs measurements, abbreviated physical examinations, slit-lamp biomicroscopy, intraocular pressure (IOP) and dilated ophthalmoscopy. Pharmacodynamic and biological activity is measured by means of BCVA by ETDRS visual acuity chart, spectral-domain optical coherence tomography (sdOCT), color fundus photography (CFP), fundus fluorescein angiography (FA), fundus autofluorescence (FAF), contrast sensititivy, and microperimetry. Pharmacokinetic (PK) and immunogenicity is evaluated by means of measuring plasma concentrations of hI-con1 and anti-drug antibodies.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A method for treating wet age-related macular degeneration (AMD) in an eye of a patient in need thereof, comprising, administering to the patient in multiple dosing sessions, a composition comprising an effective amount of an immunoconjugate dimer, wherein the monomer subunits of the dimer each comprises a mutated human factor VIIa (fVIIa) protein conjugated to the human immunoglobulin G1 (IgG1) Fc domain.

2. The method of claim 1, wherein treating the wet AMD comprises preventing, inhibiting or reversing choroidal neovascularization in the eye of the patient in need of treatment.

3. A method for preventing, inhibiting or reversing ocular neovascularization in an eye of a patient in need thereof, comprising, administering to the patient in multiple dosing sessions, a composition comprising an effective amount of an immunoconjugate dimer, wherein the monomer subunits of the dimer each comprises a mutated human factor VIIa (fVIIa) protein conjugated to the human immunoglobulin G1 (IgG1) Fc domain.

4. A method for reversing tumor neovascularization in a patient in need thereof, comprising, administering to the patient in multiple dosing sessions a composition comprising an effective amount of an immunoconjugate dimer, wherein the monomer subunits of the dimer each comprises a mutated human factor VIIa (fVIIa) protein conjugated to the human immunoglobulin G1 (IgG1) Fc domain.

5. The method of any one of claims 1-4, wherein the immunoconjugate dimer is a homodimer.

6. The method of any one of claims 1-4, wherein the immunoconjugate dimer is a heterodimer.

7. The method of claim 5 or 6, wherein at least one of the monomer subunits of the immunoconjugate comprises a mutated human fVIIa domain comprising a single point mutation at Lys341 or Ser344.

8. The method of claim 7, wherein the single point mutation is to an Ala residue.

9. The method of claim 8, wherein the single point mutation is Lys341 to Ala341.

10. The method of claim 8, wherein the single point mutation is Ser344 to Ala344.

11. The method of claims 3 and 5-10, wherein the ocular neovascularization is associated with proliferative diabetic retinopathy, wet age-related macular degeneration (AMD), retinopathy of prematurity (ROP), or neovascular glaucoma.

12. The method of any one of claims 3 and 5-10, wherein the ocular neovascularization is secondary to proliferative diabetic retinopathy, wet age-related macular degeneration (AMD), retinopathy of prematurity (ROP), or neovascular glaucoma.

13. The method of any one of claims 3 and 5-10, wherein the ocular neovascularization is choroidal neovascularization.

14. The method of claim 13, wherein the patient has been previously diagnosed with wet age-related macular degeneration (AMD) in the eye.

15. The method of claim 13, wherein the choroidal neovascularization is secondary to wet AMD.

16. The method of claim 14 or 15, wherein the eye of the patient has not been previously treated for choroidal neovascularization or wet AMD.

17. The method of claim 14 or 15, wherein the patient has previously been treated for choroidal vascularization with anti-vascular endothelial growth factor (VEGF) therapy, laser therapy or surgery.

18. The method of any one of claims 1-3 and 5-17, wherein administering comprises intravitreal injection of the composition at each dosing session.

19. The method of any one of claims 1-3 and 5-17, wherein administering comprises suprachoroidal injection of the composition at each dosing session.

20. The method of any one of claims 1-19, wherein the multiple dosing sessions comprise two or more, three or more, four or more or five or more dosing sessions.

21. The method of any one of claims 1-20, wherein each dosing session is spaced apart by from about 20 days to about 50 days, or from about 20 days to about 40 days, or from about 20 days to about 30 days.

22. The method of any one of claim 20 or 21, wherein the multiple dosing sessions comprise 12 to 24 dosing sessions.

23. The method of any one of claims 20-22, wherein administering comprises intravitreal injection of the composition into the eye of the patient once every 28 days, once every 30 days or once every 35 days.

24. The method of any one of claims 1-5 and 7-23, wherein the immunoconjugate comprises the amino acid sequence of SEQ ID NO: 2 or 3.

25. The method of claim 24, wherein the immunoconjugate comprises the amino acid sequence of SEQ ID NO: 2.

26. The method of claim 24, wherein the immunoconjugate comprises the amino acid sequence of SEQ ID NO: 3.

27. The method of claim 24, wherein the immunoconjugate is encoded by a polynucleotide sequence comprising SEQ ID ID NO:4.

28. The method of claim 24, wherein the immunoconjugate is encoded by a polynucleotide sequence comprising SEQ ID NO:5.

29. The method of any one of claims 4-10 and 20-28, wherein administering comprises intravenous administration.

30. The method of any one of claims 4-10 and 20-28, wherein administering comprises intratumoral injection.

31. The method of any one of claims 1-3 and 5-28, wherein the patient substantially maintains his or her vision subsequent to the multiple dosing sessions, as measured by losing fewer than 15 letters in a best-corrected visual acuity (BCVA) measurement, compared to the patient's BCVA measurement prior to the multiple dosing sessions.

32. The method of any one of claims 1-3 and 5-28, wherein the patient experiences an improvement in vision subsequent to the multiple dosing sessions, as measured by gaining 15 letters in a best-corrected visual acuity (BCVA) measurement, compared to the patient's BCVA prior to the multiple dosing sessions.

33. The method of any one of claims 1-3, 5-28 and 31-32, wherein subsequent to the multiple dosing sessions, or one or more of the dosing sessions, as measured by fluorescein angiography or optical coherence tomography, the CNV area is reduced in the eye of the patient, as compared to the CNV area prior to the initiation of treatment.

34. The method of claim 33, wherein the CNV area is reduced by at least about 10%, at least about 20%, at least about 30%, at least about 40% or at least about 50%.

35. The method of any one of claims 1-3, 5-28 and 31-34, wherein subsequent to the multiple dosing sessions, or a subset thereof, the retinal thickness of the eye of the patient is reduced in the eye of the patient, as compared to the retinal thickness of the eye prior to the initiation of treatment.

36. The method of claim 34, wherein the retinal thickness is reduced by at least about 50 μm, at least about 100 μm, at least about 150 μm, at least about 175 μm, at least about 200 μm, at least about 225 μm or at least about 250 μm.

37. The method of claim 34, wherein the retinal thickness is reduced by at least about 10%, at least about 20%, at least about 30%, at least about 40% or at least about 50%.

38. The method of any one of claims 35-37, wherein the decreased retinal thickness is decreased central retinal subfield thickness (CST), decreased center point thickness (CPT), or decreased central foveal thickness (CFT).

39. The method of any one of claims 18-28 and 31-38, further comprising measuring the intraocular pressure (IOP) in the eye of the patient prior to each intravitreal or suprachoroidal injection.

40. The method of any one of claims 18-28 and 31-39, further comprising measuring the IOP in the eye of the patient about 20 minutes, about 30 minutes, about 40 minutes, about 50 minutes or about 1 hour after each intravitreal or suprachoroidal injection.

41. The method of claim 39, comprising measuring the IOP in the eye of the patient about 20 minutes, about 30 minutes, about 40 minutes, about 50 minutes or about 1 hour prior to each intravitreal or suprachoroidal injection.

42. The method of any one of claims 39-41, wherein the IOP is measured via tonometry.

43. The method of any one of claims 1-42, further comprises administering an effective amount of a neovascularization inhibitor or an angiogenesis inhibitor to the patient.

44. The method of claim 43, wherein the neovascularization inhibitor or the angiogenesis inhibitor is present in the same composition as the effective amount of the immunoconjugate.

45. The method of claim 43, wherein the neovascularization inhibitor or the angiogenesis inhibitor is present in a different composition than the effective amount of the immunoconjugate.

46. The method of any one of claims 43-45, wherein the neovascularization inhibitor is a vascular endothelial growth factor (VEGF) inhibitor, a VEGF receptor inhibitor, a platelet derived growth factor (PDGF) inhibitor or a PDGF receptor inhibitor.

47. The method of claim 46, wherein the neovascularization inhibitor is ranibizumab.

48. The method of claim 47, wherein the dosage of ranibizumab is from about 0.2 mg to about 1 mg.

49. The method of claim 47 or 48, wherein the dosage of ranibizumab is 0.3 mg or 0.5 mg.

50. The method of any one of claims 7-49, wherein ranibizumab is administered to the eye of the patient via an intravitreal injection.

51. The method of any one of claims 43-50, wherein the composition comprising the effective amount of the neovascularization inhibitor or the angiogenesis inhibitor is administered to the eye of the patient via an intravitreal injection.

52. The method of claim 51, wherein the composition comprising the effective amount of the neovascularization inhibitor is administered at each of the multiple dosing sessions.

53. The method of any one of claim 18-22, wherein each dosing session comprises the administration of between about 200 μg and about 400 μg of the immunoconjugate dimer.

54. The method of claim 53, wherein the administration is about 300 μg of the immunoconjugate dimer.


SEQUENCE LISTING (5 sequences)

SEQ ID NO: 1 - DNA, 2072 bp, Artificial Sequence
  Human factor VII active site mutant immunoconjugate mRNA, complete cds
aagctttgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggtgaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaagccccaagggcgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctcggggagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcgcgggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggatccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagcggccgc

SEQ ID NO: 2 - protein, 641 aa, Artificial Sequence
  Human factor VII mutant immunoconjugate AA sequence
  MOD_RES (6)..(6): May be glutamic acid or a post translationally modified gamma carboxy-glutamic acid (GLA)
  MOD_RES (7)..(7): May be glutamic acid or a post translationally modified gamma carboxy-glutamic acid (GLA)
  MOD_RES (14)..(14): May be glutamic acid or a post translationally modified gamma carboxy-glutamic acid (GLA)
  MOD_RES (16)..(16): May be glutamic acid or a post translationally modified gamma carboxy-glutamic acid (GLA)
  MOD_RES (19)..(19): May be glutamic acid or a post translationally modified gamma carboxy-glutamic acid (GLA)
  MOD_RES (20)..(20): May be glutamic acid or a post translationally modified gamma carboxy-glutamic acid (GLA)
  MOD_RES (25)..(25): May be glutamic acid or a post translationally modified gamma carboxy-glutamic acid (GLA)
  MOD_RES (26)..(26): May be glutamic acid or a post translationally modified gamma carboxy-glutamic acid (GLA)
  MOD_RES (29)..(29): May be glutamic acid or a post translationally modified gamma carboxy-glutamic acid (GLA)
  MOD_RES (34)..(34): May be glutamic acid or a post translationally modified gamma carboxy-glutamic acid (GLA)
  CARBOHYD (52)..(52): May be site of O-glycosylation
  CARBOHYD (60)..(60): May be site of O-glycosylation
  CARBOHYD (145)..(145): May be site of N-glycosylation
  CARBOHYD (322)..(322): May be site of N-glycosylation
  CARBOHYD (491)..(491): May be site of N-glycosylation
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 641 aa, Artificial Sequence
  Human factor VII mutant immunoconjugate AA sequence
  MOD_RES (6)..(6): May be glutamic acid or a post translationally modified gamma carboxy-glutamic acid (GLA)
  MOD_RES (7)..(7): May be glutamic acid or a post translationally modified gamma carboxy-glutamic acid (GLA)
  MOD_RES (14)..(14): May be glutamic acid or a post translationally modified gamma carboxy-glutamic acid (GLA)
  MOD_RES (16)..(16): May be glutamic acid or a post translationally modified gamma carboxy-glutamic acid (GLA)
  MOD_RES (19)..(19): May be glutamic acid or a post translationally modified gamma carboxy-glutamic acid (GLA)
  MOD_RES (20)..(20): May be glutamic acid or a post translationally modified gamma carboxy-glutamic acid (GLA)
  MOD_RES (25)..(25): May be glutamic acid or a post translationally modified gamma carboxy-glutamic acid (GLA)
  MOD_RES (26)..(26): May be glutamic acid or a post translationally modified gamma carboxy-glutamic acid (GLA)
  MOD_RES (29)..(29): May be glutamic acid or a post translationally modified gamma carboxy-glutamic acid (GLA)
  MOD_RES (34)..(34): May be glutamic acid or a post translationally modified gamma carboxy-glutamic acid (GLA)
  CARBOHYD (52)..(52): May be site of O-glycosylation
  CARBOHYD (60)..(60): May be site of O-glycosylation
  CARBOHYD (145)..(145): May be site of N-glycosylation
  CARBOHYD (322)..(322): May be site of N-glycosylation
  CARBOHYD (491)..(491): May be site of N-glycosylation
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDAGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 2057 bp, Artificial Sequence
  Coding sequence for human factor VII mutant immunoconjugate AA sequence
ggatccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgagcggccgc

SEQ ID NO: 5 - DNA, 2057 bp, Artificial Sequence
  Coding sequence for human factor VII mutant immunoconjugate AA sequence
tctagaccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgaaccggt